SEQUENCE LISTING (28 sequences)

SEQ ID NO: 1 - DNA, 107 bp, Artificial
  Synthetic oligonucleotide
acgtaccaatttttgatgatgaacttcatatcctgagttatgtcggaattctgcatccatcttcacttcagagatctcctccgtcttgatatttgtcaacccagaac

SEQ ID NO: 2 - DNA, 23 bp, Artificial
  Synthetic oligonucleotide
ataanacntcttacanannccaa

SEQ ID NO: 3 - DNA, 107 bp, Artificial
  Synthetic oligonucleotide
gttctggtttgacaaatatcaagacggaggagatctctgaagtgaagatggatgcagaattccgacataactcaggatatgaagttcatcatcaaaaattggtacgt

SEQ ID NO: 4 - DNA, 12 bp, Artificial
  Synthetic oligonucleotide
gttctggtttga

SEQ ID NO: 5 - DNA, 12 bp, Artificial
  Synthetic oligonucleotide
tcttttactttc

SEQ ID NO: 6 - DNA, 12 bp, Artificial
  Synthetic oligonucleotide
gccgcagtgtta

SEQ ID NO: 7 - DNA, 12 bp, Artificial
  Synthetic oligonucleotide
gatttatcagca

SEQ ID NO: 8 - DNA, 12 bp, Artificial
  Synthetic oligonucleotide
gctcagtggaac

SEQ ID NO: 9 - DNA, 12 bp, Artificial
  Synthetic oligonucleotide
gtaagacacgac

SEQ ID NO: 10 - DNA, 12 bp, Artificial
  Synthetic oligonucleotide
gacgagcatcac

SEQ ID NO: 11 - DNA, 12 bp, Artificial
  Synthetic oligonucleotide
cgctcactgccc

SEQ ID NO: 12 - DNA, 12 bp, Artificial
  Synthetic oligonucleotide
cctcttcgctat

SEQ ID NO: 13 - DNA, 24 bp, Artificial
  Synthetic oligonucleotide
caactggtctcaagtcagtgtaca

SEQ ID NO: 14 - DNA, 26 bp, Artificial
  Synthetic oligonucleotide
tcagtggtacttatgggccagggcaa

SEQ ID NO: 15 - DNA, 24 bp, Artificial
  Synthetic oligonucleotide
ctgggagtgggtggaggcagccag

SEQ ID NO: 16 - DNA, 25 bp, Artificial
  Synthetic oligonucleotide
cagaagctggtgtctgctgtcgcca

SEQ ID NO: 17 - DNA, 20 bp, Artificial
  Synthetic oligonucleotide
aatgtcacgcacgatttccc

SEQ ID NO: 18 - DNA, 24 bp, Artificial
  Synthetic oligonucleotide
gatgacccagatcatgtttgagac

SEQ ID NO: 19 - DNA, 26 bp, Artificial
  Synthetic oligonucleotide
caaacacttcgagcacaaggggcgtt

SEQ ID NO: 20 - DNA, 26 bp, Artificial
  Synthetic oligonucleotide
cctggagctgattgagcccctcatca

SEQ ID NO: 21 - DNA, 26 bp, Artificial
  Synthetic oligonucleotide
gtaattttgacatggttgggactctt

SEQ ID NO: 22 - DNA, 22 bp, Artificial
  Synthetic oligonucleotide
acctgctgaactggtgtatgca

SEQ ID NO: 23 - DNA, 26 bp, Artificial
  Synthetic oligonucleotide
atcttcttaagaggagctaaatagca

SEQ ID NO: 24 - DNA, 26 bp, Artificial
  Synthetic oligonucleotide
ttagcagtccatatcacattgcaaaa

SEQ ID NO: 25 - DNA, 24 bp, Artificial
  Synthetic oligonucleotide
tgtcgaagtggtggtgccggctcg

SEQ ID NO: 26 - DNA, 26 bp, Artificial
  Synthetic oligonucleotide
tccagcctgagtgcagcatgaagcta

SEQ ID NO: 27 - DNA, 22 bp, Artificial
  Synthetic oligonucleotide
tgcatacaccagttcagcaggt

SEQ ID NO: 28 - DNA, 26 bp, Artificial
  Synthetic oligonucleotide
ttcaggtaaacctcagctcaggactg

(19) United States
(12) Patent Application Publication
(10) Pub. No.: US 2005/0123937 A1
(43) Pub. Date: Jun. 9, 2005

(54) Methods for the electrochemical detection of target compounds

(76) Inventors: H. Holden Thorp, Carrboro, NC (US); Mitchell Gore, Carrboro, NC (US)

Correspondence Address: Kenneth D. Sibley;Myers Bigel Sibley & Sajovec, P.A., P.O. Box 37428, Raleigh, NC, 27627, US

(21) Appl. No.: 10/794,786

(22) Filed: Mar. 5, 2004

Related U.S. Application Data
(60) Provisional application No. 60/452,879, filed on Mar. 7, 2003.
(60) Provisional application No. 60/453,742, filed on Mar. 10, 2003.
(60) Provisional application No. 60/508,327, filed on Oct. 2, 2003.

(51) Int. Cl. C12Q 1/68; C12P 19/34; C07F 15/02

(52) U.S. Cl. 435/6; 435/91.2; 546/2

(57) ABSTRACT

The present invention concerns methods for the detection of a target nucleic acid sequence in a sample.

### RELATED APPLICATIONS

This application claims the benefit under 35 U.S.C. §119 of: U.S. Provisional Patent Application Ser. Nos. 60/508,327, filed Oct. 2, 2003; 60/453,742, filed Mar. 10, 2003; and 60/452,879, filed Mar. 7, 2003, the disclosures of all of which are to be incorporated by reference herein in their entirety.

### GOVERNMENT SUPPORT

This invention was made with U.S. Government support under grant number 1 F31 HG02520-01 from the National Institutes of Health. The Government has certain rights to this invention.

### FIELD OF THE INVENTION

The present invention concerns methods for the electrochemical detection of members of specific binding pairs.

### BACKGROUND OF THE INVENTION

The detection of individual DNA sequences in heterogenous samples of DNA provides a basis for identifying genes, DNA profiling, and novel approaches to DNA sequencing. One approach to DNA hybridization detection involves the use of surface bound DNA sequences which can be assayed using an analytical response that indicates hybridization of the surface-bound oligomer to a sequence in the heterogeneous sample. These prior analytical methods generally involve laser-induced fluorescence arising from a covalently attached label on the target DNA strand, which methods are not sensitive to single-base mismatches in the surface-bound duplex. For example, U.S. Pat. Nos. 5,143,854 and 5,405,783 to Pirrung et al.; Fodor, et al., Nature 364:555 (1993); Bains, Angew. Chem. 107:356 (1995); and Noble, Analytical Chemistry 67(5):201A (1995) propose surfaces or “chips” for this application. In an alternate method, proposed by Hall, et al., Biochem. and Molec. Bio. Inter. 32(1):21 (1994), DNA hybridization is detected by an electrochemical method including observing the redox behavior of a single stranded DNA as compared to a double stranded DNA. This technique is also not sensitive to single-base mismatches in the DNA sample.

Microarrays for the detection of nucleic acid sequences of interest are among the most important biotechnology tools being developed (D. Wang et al., Science 1998, 280, 1077-1082). The ability to detect specific sequences using an inexpensive, high-throughput system with excellent signal to noise would provide an invaluable resource in both laboratory and clinical settings. Microarrays composed of spatially distinct locations containing various oligonucleotide probes that can be hybridized to in vitro nucleic acid-containing samples can be used to determine levels of gene expression and to detect single and multiple-gene polymorphisms. Such microarrays could be used at the laboratory bench to detect genes of interest in research organisms and in the clinical laboratory to detect oncogene or tumor-marker overexpression and single-gene defects in patients.

There are a variety of particular techniques that are used to detect sequence, including mutations and SNPs. These include, but are not limited to, OLA (as well as a variation, rolling circle amplification), Invader™, single base extension methods, allelic PCR, and competitive probe analysis (e.g. competitive sequencing by hybridization; see below).

Oligonucleotide ligation amplification (“OLA”, sometimes referred to herein as the ligation chain reaction (LCR)) involves the ligation of two smaller probes into a single long probe, using the target sequence as the template. See generally U.S. Pat. Nos. 5,185,243, 5,679,524 and 5,573,907; EP 0 320 308 B1; EP 0 336 731 B1; EP 0 439 182 B1; WO 90/01069; WO 89/12696; WO 97/31256 and WO 89/09835, all of which are incorporated by reference.

A variation of OLA which can also be used for genotyping is termed “rolling circle amplification”. Rolling circle amplification utilizes a single probe that hybridizes to a target such that upon ligation of the two termini of the probe, a circular probe is formed. A primer and a polymerase is added such that the primer sequence is extended. As the circular probe has no terminus, the polymerase repeatedly extends the circular probe resulting in concatamers of the circular probe. As such, the probe is amplified. Rolling-circle amplification is generally described in Baner et al. (1998) Nuc. Acids Res. 26:5073-5078; Barany, F. (1991) Proc. Natl. Acad. Sci. USA 88:189-193; Lizardi et al. (1998) Nat. Genet. 19:225-232; Zhang et al., Gene 211:277 (1998); and Daubendiek et al., Nature Biotech. 15:273 (1997); all of which are incorporated by reference in their entirety.

Invader™ technology is based on structure-specific nucleases that cleave nucleic acids in a site-specific manner. Two probes are used: an “invader” probe and a “signaling” probe, that adjacently hybridize to a target sequence with a non-complementary overlap. The enzyme cleaves at the overlap due to its recognition of the “tail”, and releases the “tail” with a label. This can then be detected. The lnvader™ technology is described in U.S. Pat. Nos. 5,846,717; 5,614,402; 5,719,028; 5,541,311; and 5,843,669, all of which are hereby incorporated by reference.

Single base extension methods can also be used for genotyping. Single base extension utilizes a polymerase and differentially labeled dNTPs; see WO 92/15712, EP 0 371 437 B1, EP 0317 074 B1; Pastinen et al., Genome Res. 7:606-614 (1997); Syvänen, Clinica Chimica Acta 226:225-236 (1994); and WO 91/13075).

An additional method is allelic PCR. As described in Newton et al., Nucl. Acid Res. 17:2503 (1989), hereby expressly incorporated by reference, allelic PCR allows single base discrimination based on the fact that the PCR reaction does not proceed well if the terminal 3′-nucleotide is mismatched, assuming the DNA polymerase being used lacks a 3′-exonuclease proofreading activity.

PCT applications WO95/15971, PCT/US96/09769, PCT/US97/09739, PCT US99/01705, WO96/40712 and WO98/20162, all of which are expressly incorporated by reference, including electrodes, which allow for novel detection methods of nucleic acid hybridization.

U.S. Pat. Nos. 5,871,918 and 6,132,971 to Thorp et al. (See also, H. Thorp, Trends Biotechnol. 1998, 16, 117-121) describe methods and apparatus for electrically detecting a target molecule by detecting a preselected base in an oxidation-reduction reaction. The methods and apparatus disclosed therein may be used in a variety of applications, including DNA sequencing, diagnostic assays, and quantitative analysis. The methods can advantageously be implemented in a variety of different assay formats and structures, including multi-well plates, with a different assay carried out in each well.

In addition, a recent paper discusses the use of electrocatalytic detection of DNA using ruthenium complexes that spontaneously bind to nucleic acids; see LaPierre et al., Anal. Chem. 2003 75:6327 (2003).

Accordingly, it is an object of the present invention to provide methods for determining the sequence of nucleic acids utilizing electrochemical detection.

### SUMMARY OF THE INVENTION

The present invention provides a method of detecting a target nucleic acid hybridization comprising:
(a) providing an electrode (e.g., a gold electrode) having a primer oligonucleotide immobilized thereon;
(b) contacting a sample containing said target nucleic acid to said primer oligonucleotide to form a hybridized nucleic acid;
(c) elongating said primer oligonucleotide using said target nucleotide as a template with a preselected detectable nucleotide to produce an elongated oligonucleotide immobilized on said electrode, with said elongated oligonucleotide containing at least one (e.g., one, two, three, four, or more, etc.,) of said preselected detectable nucleotide;
(d) reacting said elongated oligonucleotide with a transition metal complex that oxidizes said detectable nucleotide in an oxidation-reduction reaction under conditions that cause an oxidation-reduction reaction between the transition metal complex and the detectable nucleotide, from which detectable nucleotide there is electron transfer to the transition metal complex, resulting in regeneration of the reduced form of the transition metal complex as part of a catalytic cycle;
(e) detecting said oxidation-reduction reaction;
(f) determining the presence or absence of said target nucleic acid from said detected oxidation-reduction reaction at said detectable nucleotide.

In some embodiments, the detectable nucleotide is 8-oxo-guanine or 5-aminouridine.

In some embodiments, the transition metal complex is osmium²⁺ (2,2′-bipyridine)_{3.}

In some embodiments, the elongating step is carried out by asymmetric polymerase chain reaction.

A further aspect of the present invention is an electrode, such as a gold electrode, having an oligonucleotide probe immobilized thereon, as described herein (e.g., through an intervening layer).

A further aspect of the present invention is a substrate such as a non-conducting or semiconductor substrate having a plurality of separate and distinct oligonucleotides or probes immobilized thereon (e.g., in the manner described herein, such as through an intervening layer) at separate and distinct locations thereon, with each of said oligonucleotides or probes having a separate and distinct electrode (such as a gold electrode) operatively associated therewith for the detection or determination of binding of a target nucleotide thereto (e.g., by the method described above).

The present invention is explained in greater detail in the following non-limiting examples.

The specification, figures and figure legends of the U.S. Provisional Application Ser. No. 60/452,879, filed March 7 entitled METHODS OF THE ELECTROCHEMICAL DETECTION OF TARGET COMPOUNDS are specifically incorporated by reference in their entirety.

The specification, figures and figure legends of the U.S. Provisional Application Ser. No. 60/453,742, filed March 10 entitled METHODS OF THE ELECTROCHEMICAL DETECTION OF TARGET COMPOUNDS are specifically incorporated by reference in their entirety.

### BRIEF DESCRIPTION OF THE DRAWINGS

FIG. 1. Cyclic Voltammograms (CVs) obtained on gold wafer electrodes modified with mixed APPas-SH/MCH monolayers and incubated in hybridization solutions containing control APPs (AS control), 5-amino-uracil-containing APPs (AS-5-AMINO-U), 8-oxo-guanine-containing APPs (AS 8-OXO-G), and PAR19 (MS CONTROL).

FIG. 2. Cyclic voltammograms of mixed monolayer probe films hybridized to different target oligonucleotides. The CV's were collected in 100 μM Os(bpy)₃²⁺, 0.1 M KCl in E-BFR, 1V/s.

FIG. 3 schematically illustrates experimental and control electrodes placed in on electrode primer extension (OEPE) solution and subjected to the OEPE cycle.

FIG. 4 shows that, with the experimental electrode, there is a two-fold increase in oxidative current in the CV after one cycle that has increased to almost three- and four-fold after ten and twenty cycles, respectively. A concurrent increase in radiochemical signal is observed.

FIG. 5 shows, when the complementary target 3 is present in the complex mixture, a 1.5-fold increase in oxidative current is observed by the twentieth cycle (experimental electrode), while no catalytic current is observed from the control electrode after twenty cycles. This indicates that even in the presence of a 1000-fold excess of noncomplementary targets, the correct target is able to hybridize to the probe/primer film and act as a template for the extension of the thiolated primers during the OEPE cycles.

FIG. 6 shows that there is a three-fold increase in radioactivity at the experimental electrode with 3 present, while at the control electrode the amount of radiation is essentially constant and never rises above 5 CPS.

FIG. 7 shows a scheme for on electrode reverse transcription (OERT) of RNA.

FIG. 8 shows increases in radioactivity for experimental, probe control, and target control electrodes for on electrode reverse transcription of RNA.

FIG. 9 shows the cyclic voltammograms obtained before and after on-electrode primer extension using DS3-SH-modified 25 μm Au electrodes exposed to the sense strands isolated from the leukocyte (B) and SUM102 Her-2 (A) RTPCR reactions.

FIG. 10 shows the same experiment as FIG. 9, but using macroelectrodes. Again, there is current enhancement in the CV using the electrode exposed to the SUM102/Her-2 RTPCR reaction (A), while the electrodes exposed to the HT29/Her-2 (C) or leukocyte/Her-2 reactions (B) show no current enhancement.

FIG. 11 illustrates on-electrode reverse transcription, as follows: (A) mixed probe/mercaptohexanol monolayer is formed on the gold electrode surface. (B) Annealing (60° C.): The thiolated oligonucleotide, acting as both probe and primer, captures its target sequence. (C) Extension (70° C.): Taq polymerase extends the primer using the target sequence as a template. (D) Denaturation (90° C.): The target sequence is heat denatured from the electrochemically labeled probe strand and is now free to anneal to a new, unextended probe during the next annealing cycle. (E) After several cycles, the target sequence has acted as a template for the extension of many probe strands.

FIG. 12 shows the percent change in the amount of charge collected (ΔQ) before and after OERT and following hybridization of the post-synthesis tag when using DS3-SH as the surface probe.

FIG. 13 shows the number of moles of cDNA synthesized on the electrode surface during OERT using DS3-SH as the surface probe.

FIG. 14 The OERT was carried out on 25 μm gold electrodes. A) The DS3-SH-modified electrodes show noticeable catalytic current when OERT is carried out using SUM102 total RNA as the target and the electrodes are incubated in 8-oxo-G labeled-PS3. B) and C) Her2/HT29 and Her2/Leukocyte, respectively show no catalytic current after incubation in PS3, indicating that no extended thiolated probes are present to bind the labeled PS3 signal probes.

FIG. 15 The OERT was carried out on 1.6 mm gold electrodes. A) The DS3-SH-modified electrodes show noticeable catalytic current when OERT is carried out using SUM102 total RNA as the target and the electrodes are incubated in 8-oxo-G labeled-PS3. B) and C) Her2/HT29 and Her 2/Leukocyte, respectively show no catalytic current after incubation in PS3, indicating that no extended thiolated probes are present to bind the labeled PS3 signal probes.

FIG. 16 depicts a simple primer extension embodiment of the invention. Electrode 5 has primer oligonucleotide 15 attached to the electrode 5 by an attachment linker 10. Target sequence 20 is hybridized to the primer 15. An enzyme, in this embodiment a polymerase, is added, along with a reagent mixture that comprises dNTPs, one of which is a preselected detectable nucleotide, preferably a nucleotide analog with a unique redox potential 45, forming an elongation oligonucleotide that contains the label 45. An oxidation-reduction reaction is then done using a transition metal complex.

FIG. 17 depicts a variation on primer extension used for SNP detection. In this case, the primer oligonucleotide 15 has an interrogation base 35. The target has a corresponding base at the detection position 40. If perfect complementarity exists between the interrogation base 35 and the detection base 40, extension will occur. If the primer oligonucleotide interrogation base 35 does not match the target detection base 41, no extension occurs. This can be done using naturally occurring preselected nucleotides (e.g. guanine), or nucleotide analogs as described herein.

FIG. 18 depicts a simple primer extension combined with a sandwich assay. In this case, the reaction proceeds as in FIG. 16, except that the target sequence 20 is removed, and a label probe 50 is added, which contains the detection nucleotide, preferably as a “tail” of nucleotide analogs with a unique redox potential. Alternatively, although not shown, the original extension can occur with the analog dNTP and the label can contain the naturally occurring dNTP, or two analogs. In a preferred embodiment, the label oligonucleotide hybridizes to the elongated portion of the elongation oligonucleotide, so as to prevent false positives.

FIG. 19 depicts a sandwich assay format when both target alleles are present in the sample. In this embodiment, a single primer oligonucleotide captures both targets, and extension is done The target is removed and label probes with interrogation bases corresponding to the different detection bases are added, each differentially labeled.

FIG. 20 depicts a SNP detection reaction using the oligonucleotide ligation assay. In this particular embodiment, a single primer oligonucleotide is used. The example shown depicts the presence of both alleles of the target, that hybridize to the primer. Ligation probes 55 and 56 are added, each terminating in a different interrogation base and each containing a different detectable nucleotide. Only if perfect complementarity exists between the interrogation base and the detection base will ligation occur. As will be appreciated by those in the art, this can be done as a non-genotyping reaction as well, wherein the ligation probe contains the detectable nucleotide. While the figure depicts the detectable nucleotides as a component of the ligation probe, they could also (as in many embodiments depicted herein) be included as a “tail” of detectable nucleotides, to allow more signal.

FIG. 21 depicts a ligation reaction scheme for genotyping based on the use of two different electrodes 5 wherein the primer oligonucleotides 15 each carry a different interrogation base 35 and 36. In this case, a single type of label 45 can be used, or multiple labels.

FIG. 22 depicts a primer extension scheme utilizing the non-specific association of cationic metal complexes such as Ru(NH₃)₆³⁺ used with an anionic metal complex.

FIG. 23 depicts a SNP extension reaction utilizing the non-specific association of cationic metal complexes such as Ru(NH₃)₆³⁺ used with an anionic metal complex, utilizing a recruiter probe 60, allowing an increase in the signal. As will be appreciated by those in the art, it is also possible to do the reaction in the absence of the recruiter probe. Similarly, the assay can be done without a SNP component.

FIG. 24 depicts a “rolling circle” amplification reaction. In this case, the primer oligonucleotide 15 is designed to bring the two ends of the target 20 together, and then the end of the primer oligonucleotide can serve as the primer for the elongation reaction. This can be done in at least two ways; in one embodiment, the elongation incorporates the preselected detection nucleotide, or, since the elongation product results in a repeating unit, label probes can also be used.

### DETAILED DESCRIPTION OF EMBODIMENTS

The terminology used in the description of the invention herein is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise.

In general, the invention relates to the use of “on-chip” elongation technologies to allow the electrochemical detection of nucleic acids. These assays may be run in a variety of formats and for a variety of purposes, including single nucleotide polymorphism (SNP) analysis, genotyping, etc. In general, the methods rely on one of two detection schemes. In a first preferred embodiment, a primer oligonucleotide immobilized on an electrode is hybridized to a target sequence, and on-chip elongation is done using any number of amplification/extension techniques, such as primer extension, ligation, etc. This step can also include thermocycling to allow a limited amount of target to be melted off an extended primer and used to extend another primer. In one embodiment, the elongation process results in the incorporation of preselected detection nucleotides into the elongation product. As outlined below, these may be nucleotide analogs that have unique redox potentials. Alternatively, the target is removed and a label probe, comprising the preselected detection nucleotides, is hybridized to the elongated portion of the extended primer. In either case, a transition metal complex mediator is then used to facilitate an oxidation-reduction reaction to allow the detection of the elongated oligonucleotide, and thus the presence of the target sequence.

Alternatively, no preselected detection nucleotides are used; rather, instead, an elongation step is followed by the association of a cationic transition metal complex that spontaneously associates with the nucleic acid (either single stranded or double stranded, depending on the assay format) and serves as an electron transfer moiety. Upon addition of a mediator, preferably but not required to be an anionic metal complex, which serves as the mediator, an oxidation-reduction reaction occurs to allow the detection of the elongated oligonucleotide, and thus the presence of the target sequence.

In a preferred embodiment, SNP detection can be done using primer extension or other elongation techniques such as ligation or “genetic bit” (a combination of extension and ligation) that rely on the fidelity of extension enzymes such as polymerases and ligases. In these embodiments, the target contains a base at a detection position, and a probe (either the primer probe or a label probe) contains a corresponding base at the interrogation position. Only if there is perfect complementarity between the two will the extension occur, as is depicted in the Figures and described below. In addition, some embodiments utilize two or more different preselected detection nucleotides, each with a unique redox potential and mediator for SNP detection.

Accordingly, the present invention provides compositions and methods for detecting the presence or absence of target nucleic acid sequences in a sample. As will be appreciated by those in the art, the sample solution may comprise any number of things, including, but not limited to, bodily fluids (including, but not limited to, blood, urine, serum, lymph, saliva, anal and vaginal secretions, perspiration and semen, of virtually any organism, with mammalian samples being preferred and human samples being particularly preferred); environmental samples (including, but not limited to, air, agricultural, water and soil samples); biological warfare agent samples; research samples (i.e. in the case of nucleic acids, the sample may be the products of an amplification reaction, including both target and signal amplification as is generally described in PCT/US99/01705, such as PCR or SDA amplification reactions); purified samples, such as purified genomic DNA, RNA, proteins, etc.; raw samples (bacteria, virus, genomic DNA, etc.; As will be appreciated by those in the art, virtually any experimental manipulation may have been done on the sample.

By “nucleic acid” or “oligonucleotide” or grammatical equivalents herein means at least two nucleotides covalently linked together. A nucleic acid of the present invention will generally contain phosphodiester bonds, although in some cases, particularly for primer oligonucleotides and label probes, as outlined below, nucleic acid analogs are included that may have alternate backbones, comprising, for example, phosphoramide (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphophoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Left. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins et al., Chem. Soc. Rev. (1995) pp169-176). Several nucleic acid analogs are described in Rawls, C & E News Jun. 2, 1997 page 35. All of these references are hereby expressly incorporated by reference. In addition, base analogs are discussed below in the context of detectable labels.

The nucleic acids may be single stranded or double stranded, as specified, or contain portions of both double stranded or single stranded sequence. The nucleic acid may be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xathanine hypoxathanine, and those base analogs described below as detectable labels.

The compositions and methods of the invention are directed to the detection of target sequences. The term “target sequence” or “target nucleic acid” or grammatical equivalents herein means a nucleic acid sequence on a single strand of nucleic acid. The target sequence may be a portion of a gene, a regulatory sequence, genomic DNA, cDNA, RNA including mRNA and rRNA, or others. As is outlined herein, the target sequence may be a target sequence from a sample, or a secondary target such as a product of an amplification reaction, generally termed “amplicons” herein. It may be any length, with the understanding that longer sequences are more specific. As will be appreciated by those in the art, the complementary target sequence may take many forms. For example, it may be contained within a larger nucleic acid sequence, i.e. all or part of a gene or mRNA, a restriction fragment of a plasmid or genomic DNA, among others. As is outlined more fully below, probes are made to hybridize to target sequences to determine the presence or absence of the target sequence in a sample. Generally speaking, this term will be understood by those skilled in the art. The target sequence may also be comprised of different target domains; for example, a first target domain of the sample target sequence may hybridize to a primer oligonucleotide (sometimes referred to herein as a “capture probe”) and a second target domain may hybridize to all or a portion of a label probe. The target domains may be adjacent or separated as indicated. Unless specified, the terms “first” and “second” are not meant to confer an orientation of the sequences with respect to the 5′-3′ orientation of the target sequence. For example, assuming a 5′-3′ orientation of the complementary target sequence, the first target domain may be located either 5′ to the second domain, or 3′ to the second domain.

If required, the target sequence is prepared using known techniques. For example, the sample may be treated to lyse the cells, using known lysis buffers, electroporation, etc., with purification and/or amplification as needed, as will be appreciated by those in the art. Suitable amplification techniques are outlined in PCT US99/01705, hereby expressly incorporated by reference. In addition, techniques to increase the amount or rate of hybridization can also be used; see for example WO 99/67425, hereby incorporated by reference.

In as much as the processes of the present invention involve contacting the DNA sample to a primer oligonucleotide to produce a hybridized DNA for subsequent elongation and detection, it may be desirable for certain applications to amplify the DNA prior to contacting with the electrode, particularly in cases where the target concentration is low. Alternatively, as is more fully described below, an “on-chip” amplification can be done, such that multiple primer oligonucleotides are elongated using a single target sequence.

Amplification of a selected, or target, nucleic acid sequence may be carried out by any suitable means. See generally D. Kwoh and T. Kwoh, Am. Biotechnol. Lab. 8,14-25 (1990). Examples of suitable amplification techniques include, but are not limited to, polymerase chain reaction (including, for RNA amplification, reverse-transcriptase polymerase chain reaction), ligase chain reaction, strand displacement amplification, transcription-based amplification (see D. Kwoh et al., Proc. Natl. Acad. Sci. USA 86, 1173-1177 (1989)), self-sustained sequence replication (or “3SR”) (see J. Guatelli et al., Proc. Natl. Acad. Sci. USA 87, 1874-1878 (1990)), the Q.beta. replicase system (see P. Lizardi et al., Biotechnology 6, 1197-1202 (1988)), nucleic acid sequence-based amplification (or “NASBA”) (see R. Lewis, Genetic Engineering News 12 (9), 1 (1992)), the repair chain reaction (or “RCR”) (see R. Lewis, supra), and boomerang DNA amplification (or “BDA”) (see R. Lewis, supra). The bases incorporated into the amplification product may be natural or modified bases (modified before or after amplification), and the bases may be selected to optimize subsequent electrochemical detection steps. Techniques for amplification are known and described in, among other things, U.S. Pat. Nos. 4,683,195; 4,683,202; 4,800,159; and 4,965,188; G. Walker et al., Proc. Natl. Acad. Sci. USA 89, 392-396 (1992); G. Walker et al., Nucleic Acids Res. 20, 1691-1696 (1992); R. Weiss, Science 254, 1292 (1991).

In a preferred embodiment of the invention amplification is carried out by asymmetric polymerase chain reaction, in accordance with known techniques or variations thereof which will be apparent to those skilled in the art, s, e.g., Loh et al., Science, 243 (4888): 217-220 (Jan. 13, 1989)(PCR with primer attached to a surface); U.S. Pat. No. 5,075,216; U.S. Pat. No. 6,391,546.

Furthermore, preferred embodiments that use a pre-chip amplification method also preferably generate either an excess of the target strand (“Watson”) or remove the non-target strand (“Crick”) prior to contacting with the electrode.

As is more fully outlined below, in some preferred embodiments, the target sequence comprises a position for which sequence information is desired, generally referred to herein as the “detection position” which has a “detection base”. In a preferred embodiment, the detection position is a single nucleotide, although in some embodiments, it may comprise a plurality of nucleotides, either contiguous with each other or separated by one or more nucleotides. By “plurality” as used herein is meant at least two. As used herein, the base which basepairs with the detection position base in a hybrid is termed the “interrogation base” of the “interrogation position”.

Accordingly, in a preferred embodiment, the compositions and methods of the present invention are used to identify the nucleotide(s) at a detection position within the target sequence.

In a preferred embodiment, variations in temperature are used to determine either the identity of the nucleotide(s) at the detection position or the presence of a mismatch. As a preliminary matter, the use of temperature to determine the presence or absence of mismatches in double stranded hybrids comprising a single stranded target sequence and a probe is well known. As is known in the art, differences in the number of hydrogen bonds as a function of basepairing between perfect matches and mismatches can be exploited as a result of their different Tₘs (the temperature at which 50% of the hybrid is denatured). Accordingly, a hybrid comprising perfect complementarity will melt at a higher temperature than one comprising at least one mismatch, all other parameters being equal. (It should be noted that for the purposes of the discussion herein, all other parameters (i.e. length of the hybrid, nature of the backbone (i.e. naturally occurring or nucleic acid analog), the assay solution composition and the composition of the bases, including G-C content are kept constant). However, as will be appreciated by those in the art, these factors may be varied as well, and then taken into account.) In this embodiment, interrogation position is within the primer oligonucleotide, and the extension reaction is done at a temperature at which the imperfect matched hybrids are not stable.

It should be noted in this context that “mismatch” is a relative term and meant to indicate a difference in the identity of a base at a particular position, termed the “detection position” herein, between two sequences. In general, sequences that differ from wild type sequences are referred to as mismatches. However, particularly in the case of SNPs, what constitutes “wild type” may be difficult to determine as multiple alleles can be relatively frequently observed in the population, and thus “mismatch” in this context requires the artificial adoption of one sequence as a standard. Thus, for the purposes of this invention, sequences are referred to herein as “perfect match” and “mismatch”.

The present invention provides compositions comprising solid supports comprising one or more electrodes having primer oligonucleotides attached. By “solid support” or other grammatical equivalents herein is meant any material that can be modified to contain discrete individual electrodes appropriate for the attachment or association of nucleic acids. Suitable examples include, but are not limited to, glass and modified or functionalized glass, fiberglass, teflon, ceramics, mica, plastic (including acrylics, polystyrene and copolymers of styrene and other materials, polypropylene, polyethylene, polybutylene, polyimide, polycarbonate, polyurethanes, Teflon™, and derivatives thereof, etc.), GETEK (a blend of polypropylene oxide and fiberglass), polyimide, KAPTON™, etc., polysaccharides, nylon or nitrocellulose, resins, silica or silica-based materials including silicon and modified silicon, carbon, metals, inorganic glasses and a variety of other polymers, with printed circuit board (PCB) materials and microtiter plates (e.g. with individual arrays in each well) being particularly preferred.

The present system finds particular utility in array formats, i.e. wherein there is a matrix of addressable detection electrodes (herein generally referred to “pads”, “addresses” or “micro-locations”). By “array” herein is meant a plurality of primer oligonucleotides in any array format; the size of the array will depend on the composition and end use of the array. Arrays containing from about 2 different electrodes to many thousands can be made. Generally, the array will comprise from two to as many as 100,000 or more, depending on the size of the electrodes, as well as the end use of the array. Preferred ranges are from about 2 to about 10,000, with from about 5 to about 1000 being preferred, and from about 5-10 to about 30-50 being particularly preferred. In some embodiments, the compositions of the invention may not be in array format; that is, for some embodiments, compositions comprising a single electrode may be made as well. In addition, in some arrays, multiple substrates may be used, either of different or identical compositions. Thus for example, large arrays may comprise a plurality of smaller substrates.

The solid support comprises at least one, and preferably an array of electrodes. By “electrode” herein is means a composition, which, when connected to an electronic device, is able to sense a current or charge and convert to a signal. Alternatively an electrode can be defined as a composition which can apply a potential to and/or pass electrons to or from species in the solution. Thus, an electrode is an ETM as described herein. Preferred electrodes are known in the art and include, but are not limited to, certain metals and their oxides, including gold; platinum; palladium; silicon; aluminum; metal oxide electrodes including platinum oxide, titanium oxide, tin oxide, indium tin oxide, palladium oxide, silicon oxide, aluminum oxide, molybdenum oxide (Mo₂O₆), tungsten oxide (WO₃) and ruthenium oxides; and carbon (including glassy carbon electrodes, graphite and carbon paste). Preferred electrodes include gold, silicon, carbon and metal oxide electrodes, with gold being particularly preferred.

The electrodes described herein as depicted as a flat surface, which is only one of the possible conformations of the electrode and is for schematic purposes only. The conformation of the electrode will vary with the detection method used and the configuration of the system. For example, flat planar electrodes may be preferred in many systems, for example when arrays of nucleic acids are made, thus requiring addressable locations for both synthesis and detection. Alternatively, for single or low density analysis, the electrode may be in the form of a tube; this allows a maximum of surface area containing the nucleic acids to be exposed to a small volume of sample.

The electrode(s) comprise an immobilized primer oligonucleotide. By “primer oligonucleotide” or “capture probe” herein is meant a nucleic acid that hybridizes to a first domain of a target sequence, thus forming an assay complex (sometimes also referred to as a “hybridization complex”). As is known in the art, the length of the primer oligonucleotide will depend on a variety of factors, including temperature of the assay, required specificity, etc., with primer oligonucleotides generally ranging from 8 to 50 nucleotides in length, with from 10-25 being particularly preferred.

The primer oligonucleotides of the invention can be immobilized in a wide variety of ways, as will be appreciated by those in the art, depending on the composition of the electrode and the desired system. In a preferred embodiment, the primer oligonucleotide is attached using an attachment linker, which generally comprises an alkyl or aryl linker.

By “alkyl group” or grammatical equivalents herein is meant a straight or branched chain alkyl group, with straight chain alkyl groups being preferred. If branched, it may be branched at one or more positions, and unless specified, at any position. The alkyl group may range from about 1 to about 30 carbon atoms (C1-C30), with a preferred embodiment utilizing from about 1 to about 20 carbon atoms (C1-C20), with about C1 through about C12 to about C15 being preferred, and C1 to C5 being particularly preferred, although in some embodiments the alkyl group may be much larger. Also included within the definition of an alkyl group are cycloalkyl groups such as C5 and C6 rings, and heterocyclic rings with nitrogen, oxygen, sulfur or phosphorus. Alkyl also includes heteroalkyl, with heteroatoms of sulfur, oxygen, nitrogen, and silicone being preferred. Alkyl includes substituted alkyl groups. By “substituted alkyl group” herein is meant an alkyl group further comprising one or more substitution moieties “R”, as defined above.

By “aryl group” or grammatical equivalents herein is meant an aromatic monocyclic or polycyclic hydrocarbon moiety generally containing 5 to 14 carbon atoms (although larger polycyclic rings structures may be made) and any carbocylic ketone or thioketone derivative thereof, wherein the carbon atom with the free valence is a member of an aromatic ring. Aromatic groups include arylene groups and aromatic groups with more than two atoms removed. For the purposes of this application aromatic includes heterocycle. “Heterocycle” or “heteroaryl” means an aromatic group wherein 1 to 5 of the indicated carbon atoms are replaced by a heteroatom chosen from nitrogen, oxygen, sulfur, phosphorus, boron and silicon wherein the atom with the free valence is a member of an aromatic ring, and any heterocyclic ketone and thioketone derivative thereof. Thus, heterocycle includes thienyl, furyl, pyrrolyl, pyrimidinyl, oxalyl, indolyl, purinyl, quinolyl, isoquinolyl, thiazolyl, imidozyl, etc.

Suitable substitution groups include, but are not limited to, hydrogen, alkyl, aryl, include, but are not limited to, hydrogen, alkyl, alcohol, aromatic, amino, amido, nitro, ethers, esters, aldehydes, sulfonyl, silicon moieties, halogens, sulfur containing moieties, phosphorus containing moieties, and ethylene glycols.

In general, the attachment linker is tailored to the surface for attachment; for example, in the case of gold electrodes, thiol-based linkers are preferred; with indium tin oxide electrodes, silane attachments are preferred, although with some labels, such as guanine, phosphonate attachments can be used as well. In addition, in a preferred embodiment, the electrodes further comprise self-assembled monolayers (SAMs) as defined below, and in many cases the attachment linker will be the same moiety as makes up the SAM.

In order to prepare the electrode for modification with immobilized biological binding entities, the electrode is modified with a suitable intermediate layer, such as a nonconductive layer. The nonconductive layer may have one or more of a number of functions including providing covalent attachment of biomolecules, blocking of nonspecific binding to the electrode, and allowing electron transfer between the mediator and the electrode and/or the mediator and the label. The nonconductive layer may be one or more of the following, for example: self-assembled monolayers (e.g., U.S. Pat. No. 6,127,127); cross-linked polymer layers; alkyl silane layers; alkylphosphonate-, alkylphosphate-, carboxyalkane-, alkanethiol-, or alkylamine-based layers; polymer membranes (as in U.S. Pat. No. 5,968,745) and/or one or more layers of biomolecules such as proteins, antibodies, biotin-binding molecules (avidin, streptavidin, neutravidin), protein A, protein G, receptors, or oligonucleotides. In the case of a nonconductive layer comprised of biomolecules, the nonconductive layer can serve as a capture layer for the binder, target protein, the surrogate target, or the affinity ligand. For example, on an electrode designed to detect human chorionic gonadotropin (hCG), the nonconductive layer could be an anti-hCG capture antibody; on an electrode designed to detect a ligand, a receptor molecule could serve as the nonconductive layer. Alternatively, the nonconductive layer can be a biomolecule that binds the capture molecule such as protein A for a capture antibody or an antibody directed against the capture molecule (i.e. an anti-streptavidin antibody for a binding assay using streptavidin as the capture molecule or an anti-receptor antibody for a receptor-based assay). Regardless of the nature of the nonconductive layer, this layer will ultimately be placed in contact with a solution containing the mediator prior to electrochemical detection.

By “monolayer” or “self-assembled monolayer” or “SAM” herein, it is meant a relatively ordered assembly of molecules spontaneously chemisorbed on a surface, in which the molecules are oriented approximately parallel to each other and roughly perpendicular to the surface. A majority of the molecules includes a functional group that adheres to the surface, and a portion that interacts with neighboring molecules in the monolayer to form the relatively ordered array. A “mixed” monolayer comprises a heterogeneous monolayer, that is, where at least two different molecules make up the monolayer. Generally, the electrodes of the invention include a mixed monolayer, where one of the SAM forming species comprises a covalently attached primer oligonucleotide and the other does not. Species outlined above for attachment linkers are suitable for SAM species as well. In a preferred embodiment, the monolayer comprises conductive oligomers or insulators as described in PCT US01/44364, hereby incorporated by reference.

Accordingly, in a preferred embodiment, the present invention provides biochips (sometimes referred to herein “chips”) that comprise substrates comprising a plurality of electrodes, preferably gold electrodes. In some embodiments, these are in the wells of microtiter or other divided sample systems. The number of electrodes is as outlined for arrays. Each electrode preferably comprises a self-assembled monolayer as outlined herein. In a preferred embodiment, one of the monolayer-forming species comprises a capture ligand as outlined herein. In addition, each electrode has an interconnection, that is attached to the electrode at one end and is ultimately attached to a device that can control the electrode. That is, each electrode is independently addressable. See for example PCT US01/44364 hereby incorporated by reference.

The conductive substrate may take any physical form, such as an elongate shaped device having a working surface formed on one end thereof, or a flat sheet having the working surface on one side thereof, for example in the wells of a microtiter plate.

The oligonucleotides or probes may be immobilized to a substrate in accordance with-the techniques described in U.S. Pat. Nos. 6,472,148; 6,322,979; 6,197,515; and 5,620,850 (Bamdad).

Once immobilized, the primer oligonucleotide is hybridized to the target sequence to form an assay complex. The primer oligonucleotide then serves as a primer for an elongation reaction using an enzyme in a variety of formats, depending on the desired assay (straight detection, e.g. diagnosis of bacterial infections, viral infections, etc., or genotyping (e.g. SNP detection).

There are four preferred elongation methods based on the use of preselected detection nucleotides, although as will be appreciated by those in the art, other methods may be utilized. In a preferred embodiment, the elongation reaction is a primer extension elongation step. In this method, the extension enzyme is a polymerase, in some embodiments a thermostable polymerase such are known in the art, including but not limited to, DNA polymerase I, T4 DNA polymerase, T7 DNA polymerase, Taq DNA polymerase, reverse transcriptase, and RNA polymerases. The reaction mixture comprises a set of usually 4 dNTPs sufficient to elongate the primer using the hybridized target as the template. In a direct assay, one of the dNTPs is a preselected detection base, particularly a synthetic detection nucleotide with a unique redox potential. In a sandwich assay, the reaction mixture further comprises a label probe comprising the preselected detection nucleotides, such that after removal or melting off of the target (for example in the presence of excess label probe) an assay complex comprising the elongated oligonucleotide and the label probe is formed. These assays can be adapted for use for SNP detection as well, as shown in the Figures, where either the primer oligonucleotide contains a terminal base that is the interrogation position, or the label probe contains an interrogation position.

“Label probe” in this context generally refers to a nucleic acid probe that hybridizes to a second domain of the target (e.g. separate from the domain that hybridizes to the primer oligonucleotide) and contains the preselected detection nucleotides (sometimes referred to as “label nucleotides”), again, preferably analogs with unique redox potentials. Label probes can either contain the base analogs within the portion that hybridizes to the target, or contain a “tail” comprising these nucleotides, or both. In some cases, it may be desirable to utilize a label probe that has more than one detection nucleotide to increase the accuracy of the assay.

Suitable preselected detection nucleotides generally can be oxidized within a suitable voltage range for implementing the present invention, such as a range of about 0 or 0.2 volts up to about 1.4 or 1.6 volts versus a Ag/AgCl reference electrode. The labels have an oxidation potential approximately equal to or less than that of the transition metal mediator. In addition, the composition of the nonconductive layer on the electrode (e.g. the SAM) and the manner in which it adheres to the electrode may dictate the choice of detection nucleotides. For example, if a thiol-gold electrode system is used, generally detectable nucleotide analogs are used, since the voltages required for the oxidation-reduction reaction destabilize the thiol-gold attachment, and thus oxo-guanine, for example, may be preferred in this system.

Examples of suitable preselected bases include but are not limited to guanine, adenine, 8-oxo-guanine, and 8-oxo-adenine, 8-bromo-guanine, guanosine, xanthosine, wyosine, pseudouridine, 6-mercaptoguanine, 8-mercaptoguanine, 2-thioxanthine, 6-thioxanthine, 6-mercaptopurine, 2-amino-6-carboxymethyl-mercaptopurine, 2-mercaptopurine, 6-methoxypurine, 2-acetylamino-6-hydroxypurine, 6-methylthio-2-hydroxypurine, 2-dimethylamino-6-hydroxypurine, 2-hydroxypurine, 2-aminopurine, 6-amino-2-dimethylallyl-purine, 2-thioadenine, 8-bydroxyadenine, 8-methoxyadenine, 5-aminocytosine, 5-aminouridine, and 6-aminocytosine. Typically, the preselected base is selected from the group consisting of guanine, adenine, 8-oxo-guanine, 8-oxo-adenine, 7-deazaguanine, 7-deazaadenine, 5-aminocytosine, 5-aminouridine, and 6-aminocytosine, with guanine being the currently preferred naturally occurring preselected base and 7-deazaguanine the currently preferred synthetic preselected base. Preselected bases that are readily oxidized or reduced can be designed using theoretical methods described in Baik, M.-H. et al., J. Phys. Chem. B (2001),105, 6537-6444.

As noted above, the amplification process can be used to introduce synthetic preselected bases into the target by using a triphosphate of the preselected base in the amplification mixture. In general, in a primer elongation reaction, a plurality of said primer oligonucleotides are immobilized on said solid support. The hybridizing and elongating steps are then cyclically repeated by (i) releasing said target nucleic acid from said first elongated oligonucleotide; (ii) hybridizing said target nucleic acid to another of said primer oligonucleotides; and (iii) elongating said another of said primer oligonucleotides to form a subsequent elongated oligonucleotide; so that the number of elongated oligonucleotides generated for said reacting step (d) exceeds the number of said target nucleotides present in said sample. This is generally done using heat cycling as is well known in the art, and can utilize thermal cycling systems either “on-chip” or “off-chip”, e.g. when the solid support and/or reaction solution above the electrodes is contacted with a thermal cycler.

In a preferred embodiment, the elongation method is the oligonucleotide ligation assay (OLA); see generally U.S. Pat. Nos. 5,185,243 and 5,573,907; EP 0 320 308 B1; EP 0 336 731 B1; EP 0 439 182 B1; WO 90/01069; WO 89/12696; and WO 89/09835, and U.S. Ser. Nos. 60/078,102 and 60/073,011, all of which are incorporated by reference.

This method is based on the fact that certain ligation enzymes will ligate two probes together, if they are hybridized to a target strand and if perfect complementarity exists at the two bases being ligated together. Thus, in this embodiment, the target sequence comprises a contiguous first target domain comprising the detection position and a second target domain adjacent to the detection position. That is, the detection position is “between” the rest of the first target domain and the second target domain. A first ligation probe (in this case, the primer oligonucleotide) is hybridized to the first target domain and a second ligation probe is hybridized to the second target domain. If the first ligation probe (primer oligonucleotide) has a base perfectly complementary to the detection position base, and the adjacent base on the second probe has perfect complementarity to its position, a ligation structure is formed such that a ligase enzyme will ligate the two probes together to form a ligated probe. If this complementarity does not exist, no ligation structure is formed and the ligase enzyme does not ligate the probes together to an appreciable degree. As for the primer extension reaction described above, this may be done using heat cycling, to allow the release of the target sequence such that it may serve as a template for further reactions.

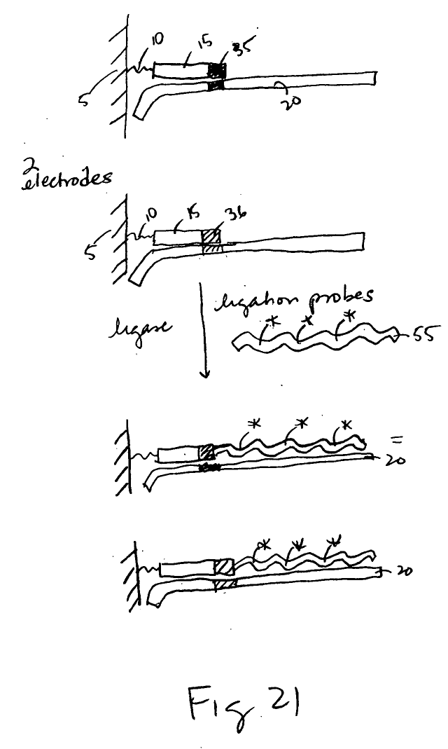

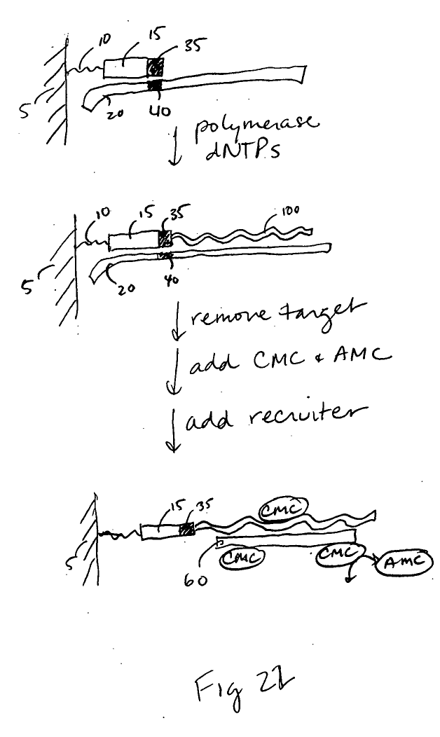

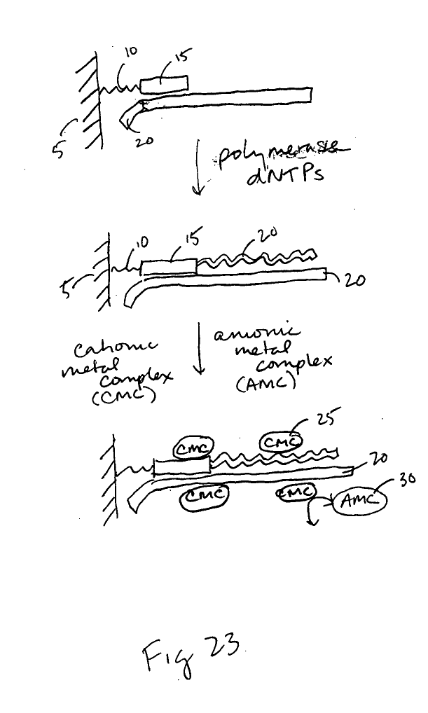

Similarly, preferred embodiments utilize the ligation elongation reaction for SNP detection, if the immobilized primer oligonucleotide has a terminal base that is the interrogation position, as is shown in FIG. 21. In this case, generally two electrodes are used, since a single ligation probe can be used.

In either embodiment, it is preferred that the ligation probe contain the preselected detection nucleotides, again, preferably analogs with unique redox potentials. Ligation probes can either contain the base analogs within the portion that hybridizes to the target, or contain a “tail” comprising these nucleotides, or both.

In a preferred embodiment, the method is a combination of extension and ligation, generally referred to in the art as “genetic bit”. In this reaction, the primer oligonucleotide serves as the first ligation probe, but there is a “gap” of at least one nucleotide that must be filled in prior to the ligation of the second ligation probe. By having either the interrogation position be the terminus of the primer oligonucleotide, the “gap”, or the terminus of the second ligation probe, and running the reaction in the presence of a polymerase and a single type of dNTP to fill in the “gap”, only if the dNTP matches the gap will the reaction proceed.

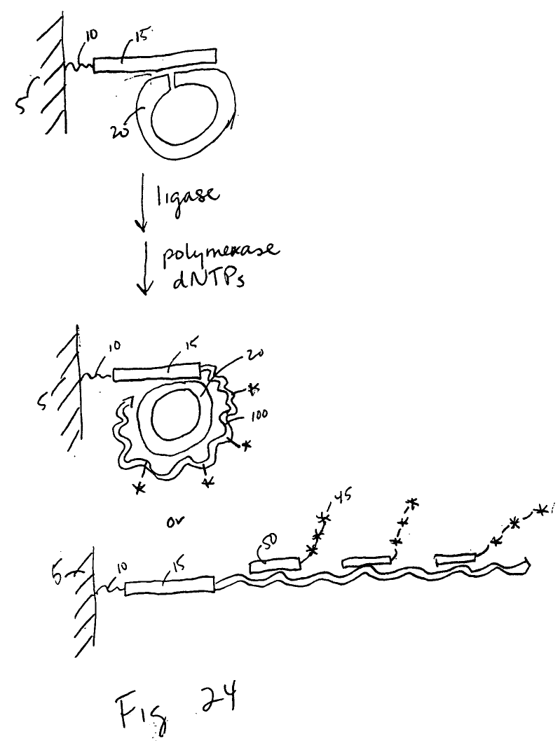

In a preferred embodiment, the elongation method is a rolling circle amplification (RCA) reaction, as generally depicted in FIG. 24. In this embodiment, a preamplification step to form an amplicon is generally, but not always, required. In this case, the primer oligonucleotide is designed to bring the two ends of the target together, so that they are adjacent, to allow the addition of a ligase to ligate the ends together to form a circle. The end of the primer oligonucleotide then serves as the primer for an extension reaction, using either dNTPs which contains the preselected detection nucleotides, or label probes that contain the detection nucleotides. As will be appreciated by those in the art, if the end of the primer oligonucleotide is the interrogation position, SNP detection can be done as no extension will occur due to the fidelity of the polymerase.

In the case where the target sequence is an RNA of any type, any of these assays can be run, with some minor modifications as will be appreciated in the art. For example, in one embodiment, the primer can be either RNA or DNA, or analogs, and a reverse transcriptase enzyme can be used, with NTPs if required, or dNTPs. In addition, some of the detectable nucleotide analogs may or may not be recognized by certain enzymes, so a tailoring of the enzyme with the detectable nucleotide of a desired redox potential can be done. In some cases, an “off chip” rtPCR can be done, and the amplicons of the reaction used as the target on the “on-chip” elongation.

Alternatively, methods that rely on the use of cationic transition metal complexes that spontaneously associate with anionic nucleic acids can be used, as is shown in the figures and outlined in the examples. Any of the methods outlined above can be used to generate elongated nucleic acids that essentially create “more” nucleic acid such that “more” cationic transition metal complexes can associate, thus increasing the signal.

“Cationic transition metal complexes” in this context must be able to serve as an electron transfer moiety and be able to spontaneously associate with the anionic backbone of the nucleic acids. Preferred cationic transition metal complexes include complexes of Ru, Co and Os, with hexamine compounds being particularly preferred. The important features of this type of label is that it form an association with nucleic acids and have a redox potential that allows its use in the desired system.

Similarly, in this embodiment, a mediator is used, as is generally defined below. In some embodiments, the mediator is an anionic metal complex; the mediator is used to regenerate the electronic status of the cationic transition metal complex. When Ru hexamine is used, a preferred mediator is ferrocyanide.

Once the elongation step is complete, and targets removed if necessary using standard techniques, a mediator is added (although as with all reagents outlined herein, they may be present at any time or in any order). A mediator used to carry out the present invention is any compound, typically a transition metal complex, that enables or makes possible electron transfer to a corresponding label as described above. In general, a different mediator will be used for each label, to which a particular mediator corresponds. The mediator may be any molecule such as a cationic, anionic, non-ionic, or zwitterionic molecule that is reactive with the electrochemical label at a unique oxidation potential to transfer electrons from the label to the electrode. It is important that the mediators used in the invention herein be selected to exhibit a reversible redox couple at about the same oxidation potential or higher than that observed for the label that is being detected. In order to use guanine as the label, the mediator must have an oxidation potential about ≧1.1 V vs. Ag/AgCl, and an appropriate mediator is Ru(bpy)₃²⁺. Other examples of suitable mediators for use in the methods of the present invention are transition metal complexes, including, for example, Ruthenium²⁺(2,2′-bipyridine)₃ (“Ru(bpy)₃²⁺”); Ruthenium²⁺(4,4″-dimethyl-2,2′-bipyridine)₃ (“Ru(Me₂-bpy)₃²⁺”); Ruthenium²⁺(5,6-dimethyl-1,10-phenanthroline)₃ (“Ru(Me₂-phen)₃²⁺”); Iron²⁺(2,2′-bipyridine)₃ (“Fe(bpy)₃²⁺”); Iron²⁺(4,4′-dimethyl-2,2′-bipyridine)₃ (“Fe(Me₂-bpy)₃²⁺”); Iron²⁺(5-chlorophenanthroline)₃ (“Fe(5-Cl-phen)₃²⁺”); Iron²⁺(4,4′-dimethyl-2,2′-bipyridine)(bipyridine)₂ (“Fe(Me₂-bpy)(bpy)₂²⁺”); Iron²⁺(4,4′-dimethyl-2,2′-bipyridine)₂(bipyridine) (“Fe(Me₂-bpy)₂(bpy)²⁺”); Osmium²⁺(2,2′-bipyridine)₃ (“Os(bpy)₃²⁺”); Osmium²⁺(4,4′-dimethyl-2,2′-bipyridine)₃ (“Os(Me₂-bpy)₃²⁺”); Osmium²⁺(5-chlorophenanthroline)₃ (“Os(5-Cl-phen)₃²⁺”); Osmium²⁺(4,4′-dimethyl-2,2′-bipyridine)(bipyridine)₂ (“Os(Me₂-bpy)(bpy)₂²⁺”); Osmium²⁺(4,4′-dimethyl-2,2′-bipyridine)₂(bipyridine) (“Os(Me₂-bpy)₂(bpy)²⁺”); dioxorhenium¹⁺phosphine; and dioxorhenium¹⁺pyridine (“ReO₂ (py)₄¹⁺”). Some anionic complexes useful as mediators are: Ru(bpy)((SO₃)₂-bpy)₂²⁻ and Ru(bpy)((CO₂)₂-bpy)₂²⁻ and some zwitterionic complexes useful as mediators are Ru(bpy)₂((SO₃)₂-bpy) and Ru(bpy)₂((CO₂)₂-bpy) where (SO₃)₂-bpy²⁻ is 4,4′-disulfonato-2,2′-bipyridine and (CO₂)₂-bpy²⁻ is 4,4′-dicarboxy-2,2′-bipyridine. Derivatives of the ferrocene molecular are also excellent mediators. Suitable substituted derivatives of the pyridine, bipyridine and phenanthroline groups may also be employed in complexes with any of the foregoing metals. Suitable substituted derivatives include but are not limited to 4-aminopyridine; 4-dimethylpyridine; 4-acetylpyridine; 4-nitropyridine; 4,4′-diamino-2,2′-bipyridine; 5,5′-diamino-2,2′-bipyridine; 6,6′-diamino-2,2′-bipyridine; 5,5′-dimethyl-2,2′-bipyridine; 6,6′-dimethyl-2,2′-bipyridine; 4,4′-diethylenediamine-2,2′-bipyridine; 5,5′-diethylenediamine-2,2′-bipyridine; 6,6′-diethylenediamine-2,2′-bipyridine; 4,4′-dihydroxyl-2,2′-bipyridine; 5,5′-dihydroxyl-2,2′-bipyridine; 6,6′-dihydroxyl-2,2′-bipyridine; 4,4′,4″-triamino-2,2′,2″-terpyridine; 4,4′,4″-triethylenediamine-2,2′,2″-terpyridine; 4,4′,4″-trihydroxy-2,2′,2″-terpyridine; 4,4′,4″-trinitro-2,2′,2″-terpyridine; 4,4′,4″-triphenyl-2,2′,2″-terpyridine; 4,7-diamino-1,10-phenanthroline; 3,8-diamino-1,10-phenanthroline; 4,7-diethylenediamine-1,10-phenanthroline; 3,8-diethylenediamine-1,10-phenanthroline; 4,7-dihydroxyl-1,10-phenanthroline; 3,8-dihydroxyl-1, 10-phenanthroline; 4,7-dinitro-1,10-phenanthroline; 3,8-dinitro-1,10-phenanthroline; 4,7-diphenyl-1,10-phenanthroline; 3,8-diphenyl-1,10-phenanthroline; 4,7-disperamine-1,10-phenanthroline; 3,8-disperamine-1,10-phenanthroline; dipyrido[3,2-a:2′,2′-c]phenazine; 4,4′-dichloro-2,2′-bipyridine; 5,5′-dichloro-2,2′-bipyridine; and 6,6′-dichloro-2,2′-bipyridine.

The mediator may be reacted with labels under conditions sufficient to effect the oxidation-reduction reaction of the mediator with the label via a catalytic reaction. The solution in which the oxidation-reduction reaction takes place may be any suitable solution for solubilizing the components of the assay and preferably comprises water. Suitable conditions for permitting the oxidation-reduction reaction to occur will be known to those skilled in the art.

The occurrence of the oxidation-reduction reaction of the invention may be detected according to any suitable means known to those skilled in the art. For example, the occurrence of the oxidation-reduction reaction may be detected using a detection (working) electrode to observe a change in the electrochemical signal, which is indicative of the occurrence of the oxidation-reduction reaction. An electrode suitable for the detection of labels in accordance with the methods described herein comprises a conductive substrate having a working surface thereon, and is sensitive to the transfer of electrons between the mediator and the label.

Generally, a reference electrode and an auxiliary electrode are also placed in contact with the mediator solution in conjunction with the detection electrode. Suitable reference electrodes are known in the art and include, for example, silver/silver chloride (Ag/AgCl) electrodes, saturated calomel electrodes (SCE), and silver pseudo reference electrodes. A suitable auxiliary electrode is a platinum electrode.

The detection of the electrochemical signal produced by the catalytic oxidation-reduction of labels permits the determination of the presence or absence of specific substances in a sample. As used herein, terms such as determining or detecting “the presence or absence” of a substance as used to describe the instant invention, also include quantitation of the amount of the substance. In the invention, the transition metal mediator is oxidized by an electrode. Then, the mediator is reduced by the label and then reoxidized at the electrode. Thus, there is electron transfer from the label to the transition metal mediator resulting in regeneration of the reduced form of the transition metal mediator as part of a catalytic cycle. The step of determining the presence or absence of target in a sample typically includes: (i) measuring the electrochemical signal generated by the oxidation-reduction reaction of the mediator at electrodes that are and are not capable of specifically binding the target, (ii) comparing the measured signal from the transition metal complex at both electrodes, and then (iii) determining whether or not the electrochemical signal generated from the mediator at the electrode that is capable of binding the target is essentially the same as, greater than, or less than, the electrochemical signal generated from the mediator at the electrode that does not bind the target. The step of measuring the electrochemical signal may be carried out by any suitable means. For example, the difference in electrochemical signal may be determined by comparing the electrochemical signal (such as current or charge) from electrodes which are and are not capable of binding the target at the same scan rate, mediator concentration, buffer condition, temperature, and/or electrochemical method.

The electrochemical signal associated with the oxidation-reduction reaction may be measured by providing a suitable apparatus in electronic communication with the detection electrode. A suitable apparatus is a potentiostat capable of measuring the electronic signal that is generated so as to provide an indication of whether or not a reaction has occurred between the label and the mediator. The electronic signal may be characteristic of any electrochemical method, including cyclic voltammetry, normal pulse voltammetry, chronoamperometry, and square-wave voltammetry, with chronoamperometry and cyclic voltammetry being the currently preferred forms.

In cyclic voltammetry, the potential of the electrochemical system is varied linearly from an initial potential between 0-800 mV to a final potential between 500-1600 mV at a constant scan rate (0.01 mV/s to 200 V/s). When the final potential is reached, the scan direction is reversed and the same potential range is swept again in the opposite direction. The preferred scan rate for Ru(bpy)₃²⁺ is 1-20 V/s with a 0 mV initial potential and a 1400 mV final potential. The current is collected at each potential and the data is plotted as a current versus potential scan. For lower-potential mediators, such as Os(bpy)₃²⁺ and Os(Me₂-bpy)₃²⁺, instead of scanning from between 0-800 mV to between 500-1600 mV, it is preferable to scan from about between 0-100 mV to between 300-1000 mV (vs. a Ag/AgCl reference electrode) because of the lower redox potentials required to oxidize these mediators.

In chronoamperometry as used in the invention herein, the electrochemical system is stepped from an initial potential between 0 mV-800 mV directly to a final potential between 500-1600 mV and held there for some specified period of time (50 μs to 10 s) and the current is collected as a function of time. If desired, the potential can be stepped back to the initial potential, and the current can be collected at the initial potential as a function of time. The preferred potential step for Ru(bpy)₃²⁺ is from between 0-800 mV to 1300 mV (vs. Ag/AgCl) with a collection time of from 50-1000 ms. For lower potential mediators, such as Os(bpy)₃²⁺ and Os(Me₂-bpy)₃²⁺, it is preferable to step from about 0-100 mV to 300-1000 mV (vs. Ag/AgCl).

In chronocoulometry, a potential step is also applied. For use in the invention herein, starting at the initial potential (0 mV-800 mV), the electrochemical system is stepped directly to the final potential (500 mV-1600 mV) (vs. Ag/AgCl). The electrochemical system is held at the final potential for some specified period of time (50 μs to 10 s) and the charge is collected as a function of time. Although not presently done, if desired, the potential can be stepped back to the initial potential and the charge can be collected at the initial potential as a function of time.

The typical apparatus that would be used for the invention herein, may, for example, include a sample container for holding a fluid sample; an electrode, as described above; and a potentiostat in electronic communication with the electrode surface. The invention may be used with a microelectronic device comprising a microelectronic substrate having first and second opposing faces, a conductive electrode on the first face, and an immobilized binder for the target substance on the second face sufficiently close to the first face to permit detection of an oxidation-reduction reaction on the second face. The oxidation-reduction reaction assay format may be in either: 1) a sandwich format wherein a target substance, captured by the immobilized first binder, is detected by a second labeled binder for the target substance, 2) a direct format wherein the target substance is captured by the immobilized first binder and is detected directly through labels bound to the target, 3) a competitive format using a labeled target or labeled surrogate target which competes with the target substance in the sample for binding to the immobilized binder, 4) a competitive format using a labeled binder and immobilized target substance with which the target substance in the sample competes for binding of the labeled binder, or 5) a binding assay format using an immobilized first binder, a second labeled binder, and a test sample which may or may not affect the interaction between the two binders.

The present invention may be carried out utilizing techniques described in, among other things, U.S. Pat. Nos. 5,871,918 and 6,132,971 to Thorp et al. Applicants specifically intend that the disclosures of all United States patent references cited herein be incorporated herein by reference in their entirety.

An electrode useful for the electrochemical detection of a preselected base in a nucleic acid in accordance with the methods described above comprises: (a) a conductive substrate having a working surface formed thereon; and (b) a nonconductive (e.g. polymer) layer connected to the working surface. The polymer layer is one that binds the primer oligonucleotides (e.g., by hydrophobic or covalent interaction or any other suitable binding technique) and is porous to the transition metal complex (i.e., the transition metal complex can migrate to the nucleic acid bound to the polymer).

An advantage of the techniques described above is that they may be carried out with a microelectronic device. A microelectronic device useful for the electrochemical detection of a nucleic acid species in the methods described above comprises a microelectronic substrate having first and second opposing faces; a conductive electrode on the first face (with or without a nonconductive layer connected thereto as described above); and an oligonucleotide capture probe immobilized on the first face adjacent the conductive electrode, or alternatively on the nonconductive layer on the electrode. The capture probe may, in addition, be spaced sufficiently close to the adjacent electrode (e.g., from about 0.1, 1, or 2.mu. up to about 50, 100, 500 or even 1000.mu.) so that an oxidation reduction reaction occurring at that probe, or at a target nucleic acid hybridized to that probe, is detected at the adjacent electrode.

In the preferred embodiment, a microelectronic device has a plurality of separate electrodes on the first opposing face, and a plurality of separate capture probes immobilized adjacent to each of the separate electrodes. By providing a plurality of separate probes, differing from one another, each with an associated electrode, a single, compact device is provided that can detect a variety of different hybridization events. Each electrode is electrically connected to a suitable contact so that the device may be wired or otherwise operatively associated with the necessary electronic equipment for carrying out the detection and determining steps of the methods described herein.

The probe may be selectively immobilized at the appropriate location on the microelectronic substrate by known techniques involving, for example, in situ synthesis of oligonucleotides on the electrode (see U.S. Pat. No. 5,405,783 to Pirrung et al.), or by deposition of probes onto the electrodes using a microarrayer (see, e.g., Guo et al., Nucl. Acids Res., 22:5456 (1994); Genetic Microsystems 417 Microarrayer). The microelectronic substrate may be a semiconductor (e.g., silicon) or non-semiconductor materials that can be processed using conventional microelectronic techniques (e.g., glass). The electrode may be metal or a non-metallic conductive material, such as polycrystalline silicon. The electrode can be formed using conventional microelectronic processing techniques, such as deposition etching. A variety of suitable microelectronic structures and fabrication techniques are well known to those skilled in the art. See, e.g., S. M. Sze, VLSI Technology (1983); S. K. Ghandhi, VLSI Fabrication Principles (1983).

In some embodiments, we utilize the methods for derivatizing gold electrodes with thiol-modified probe oligonucleotides through the formation of self-assembled monolayers (SAM's) and the hybridization of complementary target oligonucleotides to the probe strands (A. Steel et al., Anal. Chem. 1998 70, 4670-4677). This method allows time-dependent control of the density of the SAM monolayers, and the surface density of the monolayers can be determined using both electrochemical and radiolabeling methods. By incorporating modified DNA bases with oxidation potentials lower than 800 mV vs. Ag/AgCl into the target strands, one can detect the target strands on the electrode surface using a soluble metal complex mediator, Os(bpy)₃²⁺, whose oxidation potential is also lower than 800 mV vs. Ag/AgCl. When the metal complex mediator is oxidized at the electrode surface from Os(bpy)₃²⁺ to Os(bpy)₃³⁺, the oxidized mediator then associates electrostatically with the phosphate backbone of the immobilized DNA on the electrode. Since the oxidation potentials of the modified DNA bases in the target strand are approximately equal to that of the mediator, the modified bases are oxidized by the mediator, which is rereduced to Os(bpy)₃²⁺. The regenerated Os(bpy)₃²⁺ can then travel back to the electrode surface where it is then reoxidized to Os(bpy)₃³⁺, and so on. This catalytic process generates an additional 50-100% current depending upon the efficiency of the process and the hybridization efficiency of the target strand. This catalytic process then allows the detection of the electrochemically labeled target strand as a catalytic increase in the peak CV current for labeled target DNA vs. unlabeled target DNA or absent target DNA.

Amplification of a target molecule or target nucleic acid comprising an RNA is carried out in essentially the same manner as amplification of a target DNA. Any suitable mediator or transition metal complex, can be used such as described above, depending upon the choice of preselected base, with Ru(dmb)₃²⁺ preferred in one embodiment. Any suitable preselected base or preselected detectable nucleotide may be used as described above, with 7-deazaguanine currently preferred. Amplification or elongation may be carried out with any suitable reaction and reagent, with amplification with reverse transcriptase, and particularly with a thermostable reverse transcriptase, currently preferred.

The examples, which follow, are set forth to illustrate the present invention, and are not to be construed as limiting thereof. In the following examples, bp means base pair, cDNA means copy DNA, μg means microgram, ORF means open reading frame, and min means minute.

### EXAMPLE 1

Electrochemical Detection of DNA Immobilized on Gold Microelectrodes

Materials. All solutions were made with deionized water (18 mΩcm resistivity) from a Millipore MilliQ system. DNA oligonucleotides were purchased from the UNC-Chapel Hill Lineberger Comprehensive Cancer Center Nucleic Acids Core Facility (Chapel Hill, N.C.) and are listed in Table 1. The probe DNA strand used (1) was a 107-bp segment (antisense strand) of the homo sapiens amyloid precursor protein gene (chromosome 21), D678N mutant. The noncomplementary target DNA control (2) was a 23-mer sequence with 8-oxo-guanine at positions 5, 8, 16, 18, 19. Hexaammineruthenium (III) chloride (99%) was purchased from Strem Chemical and used as received. Os(bpy)₃Cl₂ was purchased from Sigma-Aldrich and used as received. 8-oxo-guanosine triphosphate was purchased from Tri-link Biotechnologies, Inc. (San Diego, Calif.) and used as received. The buffers used were the same as those used by the Tarlov group (A. Steel et al., Anal. Chem. 1998 70, 4670-4677). DNA deposition was carried out in D-BFR, 1.0 M potassium phosphate buffer, pH 7; electrode rinsing was carried out in R-BFR, 10 mM NaCl, 5 mM Tris buffer, pH 7.4; hybridization was carried out in H-BFR, 1.0 M NaCl, 10 mM Tris Buffer, pH 7.4,1 mM EDTA; electrochemical experiments were carried out in E-BFR, 10 mM Tris Buffer, pH 7.4.

TABLE 1
Oligonucleotide Sequences.

| | |
|---|---|
| 1 | 5′-ACGTACCAATTTTTGATGATGAACTTCATATCCTGAGTTATGTC |
| | GGAATTCTGCATCCATCTTCACTTCAGAGATCTCCTCCGTCTTGATA |
| | TTTGTCAACCCAGAAC-3′^{a} |
| 2 | 5′-ATAA8AC8TCTTACA8A88CCAA-3′^{b} |
| 3 | 5′-GTTCTGGTTTGACAAATATCAAGACGGAGGAGATCTCTGAAGTG |
| | AAGATGGATGCAGAATTCCGACATAACTCAGGATATGAAGTTCATCA |
| | TCAAAAATTGGTACGT-3′^{c} |
| 4 | 5′-PCB-GTTCTGGTTTGA-3′^{d} |
| 5 | pUC19 plasmid linearized sense strand |
| 6 | 5′-TCTTTTACTTTC-3′ |
| 7 | 5′-GCCGCAGTGTTA-3′ |
| 8 | 5′-GATTTATCAGCA-3′ |
| 9 | 5′-GCTCAGTGGAAC-3′ |
| 10 | 5′-GTAAGACACGAC-3′ |
| 11 | 5′-GACGAGCATCAC-3′ |
| 12 | 5′-CGCTCACTGCCC-3′ |
| 13 | 5′-CCTCTTCGCTAT-3′ |

^{a}1-SH is 1 containing a 5′-(CH₂)₆-thiol.
^{b}8 is 8-oxo-guanine.
^{c}3-8G is 3 in which all G's are replaced by 8-oxo-guanine, 3-5U is 3 in which all T's are replaced by 5-amino-uracil.
^{d}PCB is a 5′ photocleavable biotin.

Electrode preparation. Gold macroelectrodes were prepared by evaporation of a 200 angstrom chromium adhesion layer followed by a 2000 angstrom gold layer (both 99.99% purity) onto clean 1 cm×1 cm glass squares. Gold microelectrodes (10 μm and 25 μm) were purchased from BAS. Before each experiment electrodes were cleaned by immersion in warm piranha solution (70% concentrated sulfuric acid, 30% hydrogen peroxide solution(30%)) for 15 min followed by 5% aqueous hydrofluoric acid for 30 s. The electrodes were then rinsed thoroughly with deionized water and immersed in the DNA deposition solutions while still wet.

DNA SAM's were prepared using the procedure used by the Tarlov group. The clean gold macro-or-micro-electrode was immersed in a 1.0 μm solution of probe oligonucleotide (1) in D-BFR for 2 hours, rinsing with R-BFR for 5 s, immersing in a 1.0 mM 6-mercapto-1-hexanol solution (MCH) solution in deionized water for 1 h, and rinsing for 5 s with R-BFR.

Hybridization was performed at 35° C. for 60 min in H-BFR. The concentration of complementary target and noncomplementary target for nonspecific absorption controls was 0.1 μm. After removal from the hybridization solution, electrodes were rinsed with R-BFR for 5 s.

Experiments. Cyclic voltammetry (CV) and chronocoulometry (CC) were performed with a BAS100B (macroelectrodes) or Axon Geneclamp 500 amplifier/Digidata 1200 (microelectrodes) electrochemical analyzer with a single-compartment voltammetric cell equipped with a 1 cm×1 cm gold wafer macroelectrode or 10 or 25 μm diameter gold wire microelectrode as the working electrode, a Pt-wire counter electrode, and an Ag/AgCl reference electrode. The Ag/AgCl electrode was purchased from Cypress, Inc. For solutions containing Os(bpy)₃²⁺, cyclic voltammograms from 0 to 0.8 V were taken at a scan rate of 1 V/s. For solutions containing Ru(NH₃)₆³⁺, chronocoulometric response curves were collected by stepping the potential from 0.3 to −0.6V. Background scans of buffer alone were collected for both techniques and subtracted from scans of metal complexes and immobilized DNA. All experiments were carried out at laboratory ambient temperature (21-25° C.).

Synthesis of 5-NH₂-dUridine triphosphate. 5-NH₂-dUridine (Sigma-Aldrich) was phosphorylated using the procedure developed by Hoheisel and Lehrach (J. Hoheisel et al., FEBS 1990, 27, 103-106). 0.3 mmol 5-NH₂-dUridine was stirred in 750 μl dry Et₃PO under nitrogen at 0° C. 0.45 mmol proton sponge (Sigma-Aldrich) and 2 mmol POCl₃ (Strem) were added and stirred at 0° C. for 2 hours to create solution 1. In a separate flask, 2 mmol pyrophosphoric acid was dissolved in 2 mL tri-n-butylamine under nitrogen. The resulting oil was dried via coevaporation with 8 mL pyridine (2×) with 70 μL tri-n-butylamine added before the second drying step. The oil was then dissolved in 4 mL DMF to create solution 2. Solutions 1 and 2 were combined and stirred at 0° C. under nitrogen for 2 min. The reaction was then stopped by addition of 30 mL of 0.2M triethylammonium bicarbonate and kept on ice for four hours. The pH of the reaction mixture was then adjusted to 7.5 with 1 M NaOH. The solution was dried via rotovap at 45° C. and the solid was extracted with 1 volume diethyl ether and filtered. The H₂O fraction was dried via rotovap and the product was isolated on a 2.5×20 cm Q-sepharose column (bicarbonate form) using a linear gradient of 0.15 M to 0.8 M triethylammonium bicarbonate. 32.6 μmol 5-NH₂-dUridine triphosphate was isolated as a sticky brown solid (10.87% yield). The identity and purity of the triphosphate product was confirmed by ³²p NMR in D₂O (300 MHz, s, δ=4.188 vs. 85% H₃PO₄), thin-layer chromatography vs. a dNTP mix, dCMP, and the 5-NH₂-dUridine starting material (B. Bochner et al., J. B. C. 1982, 257, 9759-9769), Klenow (exo-) primer extension assay, and LC-MS.

Target Oligonucleotide Synthesis. The target oligonucleotide (3, 3-8G, 3-5U) was synthesized using a Klenow (exo-) primer extension procedure. The 107-mer template (1) was combined with the 12-mer primer (4) in a 0.01M sodium phosphate buffer solution (pH 7.0) such that both template and primer were present at a concentration of 1 mM. The solution was then heated to 90° C. for 5 min and allowed to cool to room temperature for 2 hours. The solution was then divided into three equal volumes. To the control reaction was added a solution of all four dNTPs such that the final concentration of each dNTP was 1 mM. For the 8-oxo-guanine reaction, the guanine solution was replaced with 8-oxo-guanine at a final concentration of 1 mM, and for the 5-amino-uracil reaction, the thymine solution was replaced with 5-amino-uracil at a final concentration of 1 mM. One-tenth of a solution volume of 10× Eco-Pol buffer and 2 μl 5-U Klenow (exo-) were then added to each of the three solutions. The solutions were then incubated at 37° C. for one week. The DNA in each reaction was then ethanol precipitated and dissolved in 400 μl TEN₁₀₀ binding buffer (Roche). The photocleavable-biotinylated sense strand was then isolated from the template strand using the procedure included with the Roche strepavidin magnetic particles and the magnetic particle separator (Instructions for streptavidin magnetic particles Cat. No. 1 641 778 and 1 641 786 and magnetic particle separator Cat. No. 1 641 794, version 3, October 1999, Roche Diagnostics GmbH, Roche Molecular Biochemicals). The photocleavable-biotinylated sense strand was then cleaved from the magnetic beads by irradiation with an Oriel arc Hg lamp at 300 W and a 350 nm cutoff filter. The UV light was filtered by placing a glass jar full of water between the three samples and the lamp. The magnetic beads were then removed, and the free oligonucleotide was ethanol precipitated and 5′radiolabeled. The full-length sense strand was gel purified by electrophoresis in a 20% denaturing polyacrylamide gel, the gel was phosphorimaged in order to locate the full-length band, and the full-length strand was cut out of the gel. The gel band was then shaken overnight in 1 M aqueous sodium acetate in microfuge filters, centrifuged at 14,000×g, ethanol precipitated, and dissolved in H-BFR. The amount of DNA isolated was determined by UV-Vis at 260 nm. The respective target oligonucleotides (3, 3-8G, and 3-5U) were then brought to a final concentration of 1 μM oligonucleotide in H-BFR.

Quantification of DNA. The probe DNA surface density at the electrode surface was calculated using both radiolabeling and the chronocoulometric method devised by the Tarlov group. The radiolabeling procedure entailed radiolabeling either the 5′-end (for target DNA) or the 3′-end (for probe DNA) using γ-phosphorylated ³²P dATP and polynucleotide kinase for 5′-labeling or dideoxy-³²P dATP and terminal deoxynucleotidal-transferase for 3′-labeling. The labeled, immobilized oligonucleotides on the gold electrodes were then phosphorimaged and compared to phosphorimaged control spots of known amounts of the appropriate labeled oligonucleotide solutions on filter paper, and the amount of immobilized DNA was determined using the Imagequant software package.

The Tarlov chronocoulometric method has been detailed elsewhere (A. Steel et al., Anal. Chem. 1998 70, 4670-4677). Briefly, the DNA-modified electrode is first immersed in E-BFR and a chronocoulommogram is collected by stepping the potential from 0.3 to −0.6 V for 250 ms. The electrode is then rinsed and immersed in a solution of 100 μM Ru(NH₃)₆³⁺ in E-BFR, and a chronocoulommogram is collected using the same procedure. The chronocoulometric response curves are converted to Anson plots by plotting charge vs. time^{1/2}. The linear part of the Anson plot is then extrapolated back to time zero to obtain the intercept for the plot in the presence and absence of Ru(NH₃)₆³⁺. Since Ru(NH₃)₆³⁺ in a low-ionic strength buffer essentially completely exchanges with the native charge-compensating cation of the immobilized DNA, the amount of charge-compensating Ru(NH₃)₆³⁺ trapped at the electrode surface can be measured by chronocoulometry, and the surface density of the immobilized DNA is obtained from the integrated Cottrell expression via the equations:
Q=(2nFAD₀^{1/2}C₀/π^{1/2})t^{1/2}+Q_{dl}+nFAΓ₀ (1)
Γ_{DNA}=Γ₀(z/m)(N_{A}) (2)
where Q is charge (C), t is time (s), n is the number of electrons per reduced molecule, F is the Faraday constant (C/equiv), A the electrode area (cm²), D₀ the diffusion coefficient (cm²/s), C₀ the bulk concentration (mol/cm²), Q_{dl} the capacitive charge (C), nFAΓ₀ the charge from the reduction of Γ₀ (mol/cm²) of adsorbed redox marker, m the number of bases in the probe or target DNA, z the charge of the redox molecule, N_{A} Avogadro's number, and Γ_{DNA} the surface density of the DNA in molecules/cm². The surface excess of redox marker is then calculated as the difference in the chronocoulometric intercepts in the absence and presence of Ru(NH₃)₆³⁺. The procedure was carried out after DNA probe deposition and again after DNA target hybridization on order to determine the hybridization efficiency for the experiment.

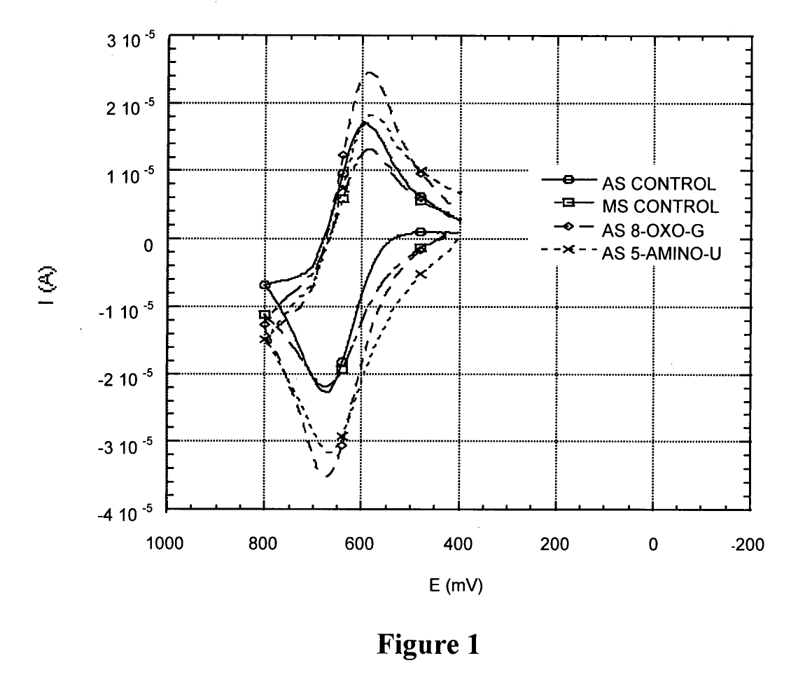

Detection of electrochemically labeled oligonucleotides. The observation of electrocatalytic current from target oligonucleotides containing 5-amino-uracil (3-5U) and 8-oxo-guanine (3-8G) was first investigated at 1 cm×1 cm gold wafer electrodes. Mixed 1-SH/MCH monolayers on gold were formed by immersion of the gold electrodes in the respective thiol solutions for 2 hours. The SAM-modified electrode was then immersed in a solution of 2, 3, 3-8G, or 3-5U. Cyclic voltammograms of the respective films were then collected in a solution of 100 μM Os(bpy)₃²⁺ and 0.01 M KCl in E-BFR, pH 7.4, at a scan rate of 1 V/s. As FIG. 1 shows, 3-8G and 3-5U produce a ˜50% peak oxidative current enhancement vs. the control target. Additionally, the missense control target 2 exhibits virtually the same current response as the sense control target, indicating that there is little, if any, non-specific adsorption to the gold surface. This is consistent with the observations of the Tarlov group, in which both chronocoulometry and X-ray photoelectron spectroscopy indicated that the diluent MCH film prevents the nonspecific adsorption of noncomplementary DNA to the MCH-modified gold surface. The averages of the peak current responses of five different films of each type were collected (3: −2.3±0.04 μA; 2: −1.74±0.08 μA; 3-8G: −3.47±0.06 μA; 3-5U: −2.87±0.1 μA) illustrating that the detection of the catalytic current for the modified-base containing films is both consistent and reproducible. The small standard errors indicate that the catalytic current is a real result of the presence of the modified bases, and not simply a result of differences in the properties of the individual films themselves.

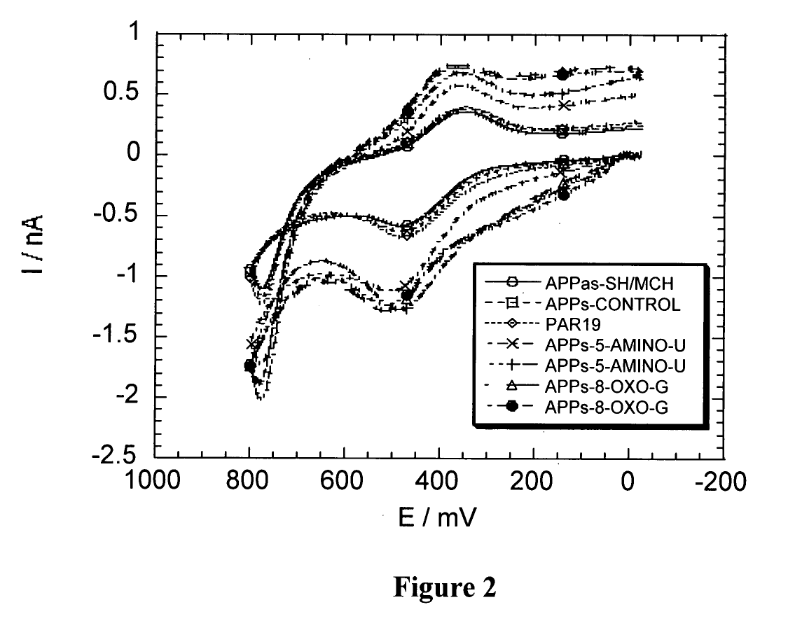

The successful detection of electrochemically-labeled nucleic acids on gold macroelectrodes led us to investigate the extension of the same method using gold wire microelectrodes. Mixed 1-SH/MCH monolayers were formed on a 25 μm gold wire electrode by immersion of the electrode in the respective thiol solutions for 2 hours. In this case, in order to investigate the stability of the DNA-modified electrode and the robustness of the electrochemical detection system, the hybridization of each subsequent type of target oligonucleotide was carried out with the same probe monolayer film, with the previous target sequence being removed by denaturation of the film at 90° C. for 5 min in 1 M sodium phosphate buffer (pH 7.4). Cyclic voltammograms of each film were then collected after each hybridization step. Since the gold-thiol bond is known to be stable above 100° C., we expected that the electrode could be regenerated by heat denaturation after each hybridization, which would mean that any current increase observed would come solely from the presence of the target strand present. As FIG. 2 shows, the CV peak current values for the mixed monolayers probe film, 2, and 3 are all virtually identical, illustrating again that there is no current enhancement for non-electrochemically-labeled target strands, and that the electrochemically-labeled noncomplementary control target does not non-specifically adsorb to the electrode surface. When the electrochemically-labeled target strands are hybridized to the probe monolayers, however, there is a greater than 50% peak CV current increase. After the electrochemically-labeled target strands are heat denatured, the catalytic current disappears, indicating that the current is solely the result of the present of the labeled oligonucleotide, and that the mixed monolayers film is indeed regenerated intact by the heat denaturation.

Quantification of immobilized DNA. Table 2 shows the probe and target surface densities calculated for the gold macroelectrodes and gold wire microelectrodes. All of the values are within the range (˜1-10×10¹² molecules/cm²) that the Tarlov group observed, and below the maximum value imposed by the physical dimensions of the DNA double helix itself (A. Steel et al., Anal. Chem. 1998 70, 4670-4677; K. Nadassy et al., NAR 2001, 29, 3362-3376). Although there is some variance between the values obtained for the macro-vs. microelectrodes, and between the values obtained using the two different methods, within errors the differences are actually relatively small, and more importantly, the hybridization efficiencies are similar for the macroelectrodes (34.5% vs. 46.6%) and nearly identical for the microelectrodes (64.5% and 69%). The differences may be due to variances in the conditions used to deposit and hybridize the DNA and the large size difference for the macro-vs. microelectrodes, as well as inherent differences in the two quantification methods.

TABLE 2
DNA Surface Densities × 10¹²

| | 1 | 3 |
|---|---|---|
| Macroelectrodes | | |
| Radiolabeling | 5.94 +/− 0.5 molecules/cm² | 2.77 +/− 0.5 |
| | | molecules/cm² |
| Chronocoulometry | 3.57 +/− 0.5 molecules/cm² | 1.23 +/− 0.1 |
| | | molecules/cm² |
| Microelectrodes | | |
| Radiolabeling | 0.81 +/− 0.1 molecules/cm² | 0.52 +/− 0.3 |
| | | molecules/cm² |
| Chronocoulometry | 2.69 +/− 0.6 molecules/cm² | 1.86 +/− 0.8 |
| | | molecules/cm² |

### EXAMPLE 2

On Electrode Primer Extension (OEPE)

BAS 1.6 mm or 25 μm Au electrodes were cleaned with piranha solution and rinsed with ddH₂O. The electrodes were then derivatized with a 5′-thiolated primer and mercaptohexanol by the usual method. The electrodes were then incubated in a solution of the complementary sequence in hybridization buffer. The OEPE solution consisted of 360 μl ddH₂O, 10 μl 30 mM MgCl₂, 1 μl each of α-³²P-dATP, dCTP, dTTP, and 8-oxo-dGTP, 1 μl Taq polymerase, and 40 μl 10×PCR buffer. The derivatized electrode was placed in the OEPE solution and incubated for 3 min. at 72° C., 3 min. at 94° C., and 55° C. for 3 min. This was repeated for 20 cycles, with Geiger counter readings and cyclic voltammograms collected after 0, 1, 10, and 20 cycles. The electrode was rinsed thoroughly with R-BFR before Geiger readings and CV's were collected.

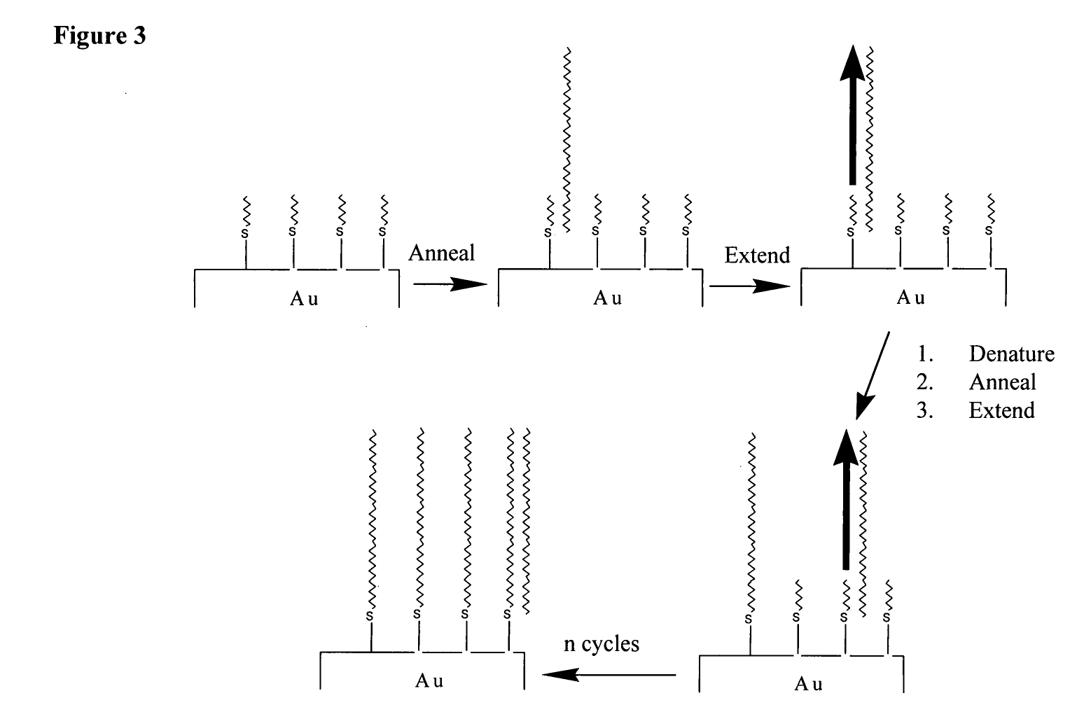

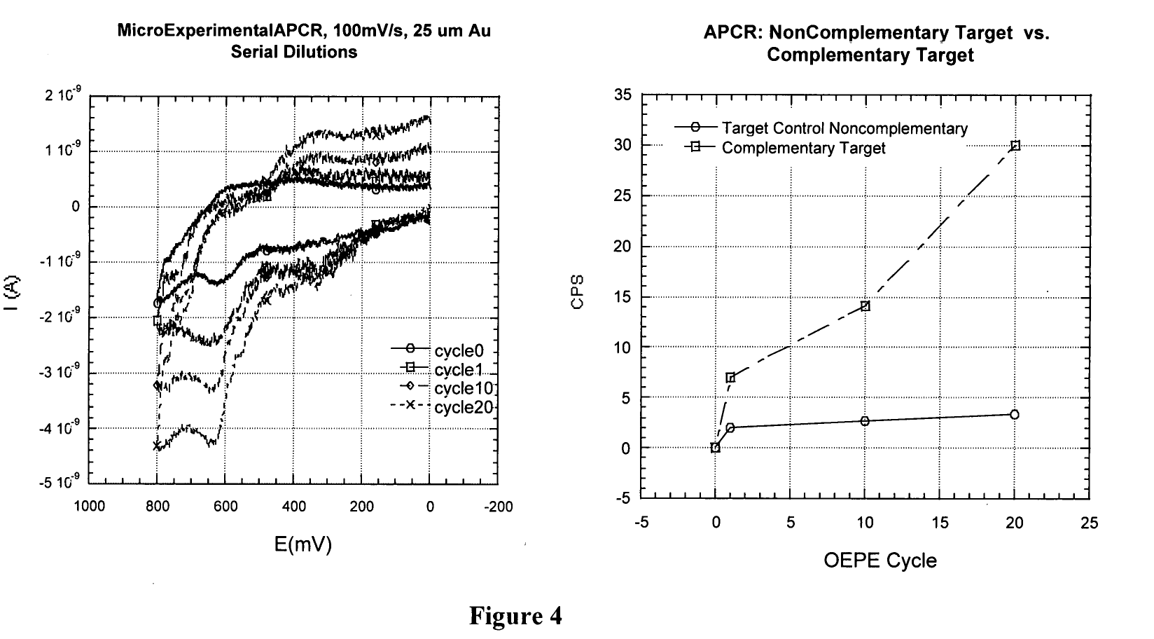

On Electrode Primer Extension at an Au Electrode. The use of OEPE to electrochemically label and indirectly detect nucleic acid in solution was investigated using a 25 μm Au electrode. A mixed 1-SH/MCH monolayer was formed using the usual procedure. A 1 μL solution containing 40 attomoles of 3 in H-BFR was prepared and placed on the inverted electrode. For the control experiment, 1 μL of H-BFR containing 40 attomoles of 1 was used. The experimental and control electrodes were then placed in the OEPE solution and subjected to the OEPE cycle detailed above and shown in FIG. 3. As FIG. 4 shows, with the experimental electrode there is a two-fold increase in oxidative current in the CV after one cycle that has increased to almost three- and four-fold after ten and twenty cycles, respectively. This illustrates that the OEPE inserts increasing numbers of electrochemically-labeled bases into the DNA synthesized as the number of cycles increases, which in turn increases the signal-to-noise ratio in the CV. FIG. 4 also shows that the amount of radiation at the electrode surface (indicated by Geiger counter counts per second) increases four-fold for the experimental electrode, from seven CPS to thirty CPS. This indicates that increasing amounts of radiolabeled dATP are incorporated into the extended thiolated primers during the OEPE. In contrast, the amount of radiation at the control electrode stays essentially unchanged at two to four CPS. This background level of radiation indicates that some dATP adheres nonspecifically to the electrode, but that no DNA synthesis takes place in the absence of the correct target sequence, even after multiple cycles.

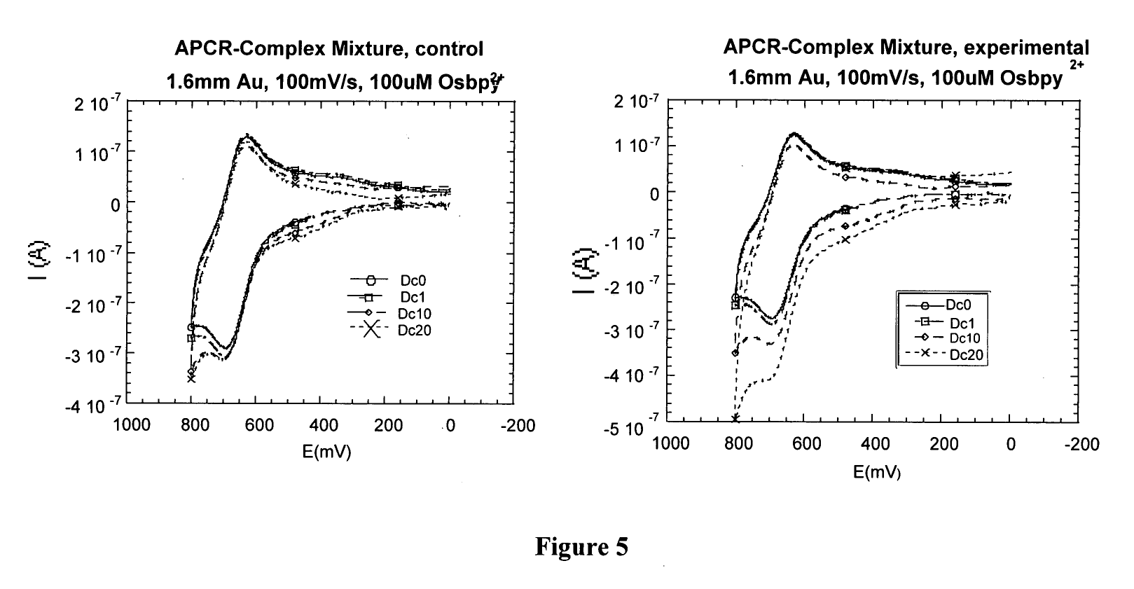

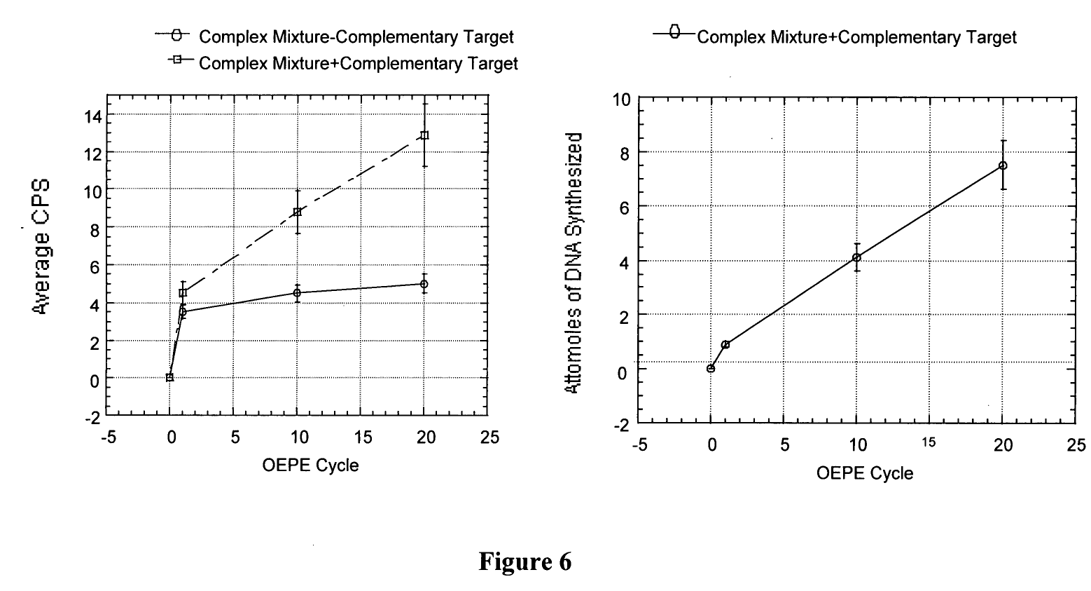

On Electrode Primer Extension using a Complex Mixture. The successful use of OEPE to electrochemically label, indirectly amplify, and detect a target sequence of interest led us to attempt to use OEPE to detect a target sequence of interest in the presence of an excess of noncomplementary targets. In this case mixed 1-SH/MCH monolayers were formed on two 1.6 mm Au electrodes using the usual procedure. The experimental electrode was then incubated in a solution containing 40 femtomoles of 3 and 40 picomoles each of 1, 2, and 5-13 in H-BFR (see Table 1). The control electrode was then incubated in a solution containing only 40 picomoles each of 1, 2, and 5-13 in H-BFR. The electrodes were then placed in OEPE solutions and subjected to the usual OEPE procedure. As FIG. 5 shows, when the complementary target 3 is present in the complex mixture, a 1.5-fold increase in oxidative current is observed by the twentieth cycle, while no catalytic current is observed from the control electrode after twenty cycles. This indicates that even in the presence of a 1000-fold excess of noncomplementary targets, the correct target is able to hybridize to the probe/primer film and act as a template for the extension of the thiolated primers during the OEPE cycles. FIG. 6 shows that there is a three-fold increase in radioactivity at the experimental electrode with 3 present, while at the control electrode the amount of radiation is essentially constant and never rises above 5 CPS. This indicates that even in a complex mixture of various sequences no DNA synthesis takes place in the absence of the correct target. After 20 cycles the amount of DNA synthesized after the first cycle has increased by almost an order of magnitude, indicating that OEPE can be a powerful tool for indirectly amplifying a rare sequence of interest in the presence of other noncomplementary sequences while concomitantly incorporating an electrochemical label into the DNA synthesized.

### EXAMPLE 3

On Electrode Primer Extension from RNA Targets

A. Materials and Methods.

All solutions were made with deionized water (18 mΩcm resistivity) from a Millipore MilliQ system. DNA oligonucleotides were purchased from the UNC-Chapel Hill Lineberger Comprehensive Cancer Center Nucleic Acids Core Facility (Chapel Hill, N.C.). The rTth themostable DNA polymerase kit was purchased from Applied Biosystems (Foster City, Calif.). Human leukocyte total RNA was isolated from whole blood using the PAXGENE blood RNA validation kit, purchased from Qiagen (Valencia, Calif.). The experimental probe DNA strand used (U. Michel et al., Analytical Biochemistry 1997, 249, 246-247) was a 24-bp downstream primer for the human β-actin gene (Genbank accession number X00351). The control probe DNA strand used (2) was a 26-bp downstream primer for the human εl-hemoglobin gene (Genbank accession number NM_{—}005330). The noncomplementary target DNA control was the pAW109 plasmid RNA included with the rTth kit. 7-deaza-2′-deoxyguanosine triphosphate was purchased from Roche (Indianapolis, Ind.). The buffers used were the same as those used by Steel et al. (Anal. Chem. 1998 70, 46704677). DNA deposition was carried out in D-BFR, 1.0 M potassium phosphate buffer, pH 7; electrode rinsing was carried out in R-BFR, 10 mM NaCl, 5 mM Tris buffer, pH 7.4; electrochemical experiments were carried out in 50 mM sodium phosphate buffer, pH 7.4, with 50 mM NaCl. Nucleic acid probes and primers used in this experiment are shown in Table 3.

Electrode Preparation. Gold microelectrodes (25 μm diameter) were purchased from Bioanalytical Systems. Before each experiment electrodes were cleaned by immersion in warm piranha solution (70% concentrated sulfuric acid, 30% hydrogen peroxide solution (30% in H₂O)) for 15 min followed by 5% aqueous hydrofluoric acid for 30 s. The electrodes were then rinsed thoroughly with deionized water and immersed in the DNA deposition solutions while still wet.

The modified electrodes were prepared using the procedure of Steel et al. (supra). The clean gold electrode was immersed in a 1.0 μM solution of probe oligonucleotide (1 or 2) in D-BFR for 1 h, rinsed with R-BFR for 5 s, immersed in a 1.0 mM 6-mercapto-1-hexanol solution (MCH) solution in deionized water for 0.5 h, and rinsed for 5 s with R-BFR.

Experiments. Cyclic voltammetry (CV) was performed with a BAS100B electrochemical analyzer with a single-compartment voltammetric cell equipped with a 25 μm diameter gold wire microelectrode as the working electrode, a Pt-wire counter electrode, and a Ag/AgCl reference electrode. The Pt-wire and Ag/AgCl electrodes were purchased from BAS, Inc. Cyclic voltammetry was performed in 50 mM sodium phosphate buffer, pH 7.4, with 50 mM NaCl with 100 mM Ru(dmbpy)₃²⁺. For solutions containing Ru(dmbpy)₃²⁺, cyclic voltammograms were taken from 0 to 1.2 V at a scan rate of 100 mV/s. All experiments were carried out at laboratory ambient temperature (21-25° C.).

TABLE 3
Oligonucleotide Sequences

| | |
|---|---|
| 1 | 5′-HS-(CH₂)₆-CAACTGGTCTCAAGTCAGTGTACA-3′ |
| 2 | 5′-HS-(CH₂)₆-TCAGTGGTACTTATGGGCCAGGGCAA-3′ |
| 3 | 5′-CTGGGAGTGGGTGGAGGCAGCCAG-3′ |
| 4 | 5′-CAGAAGCTGGTGTCTGCTGTCGCCA-3′ |

B. Experimental

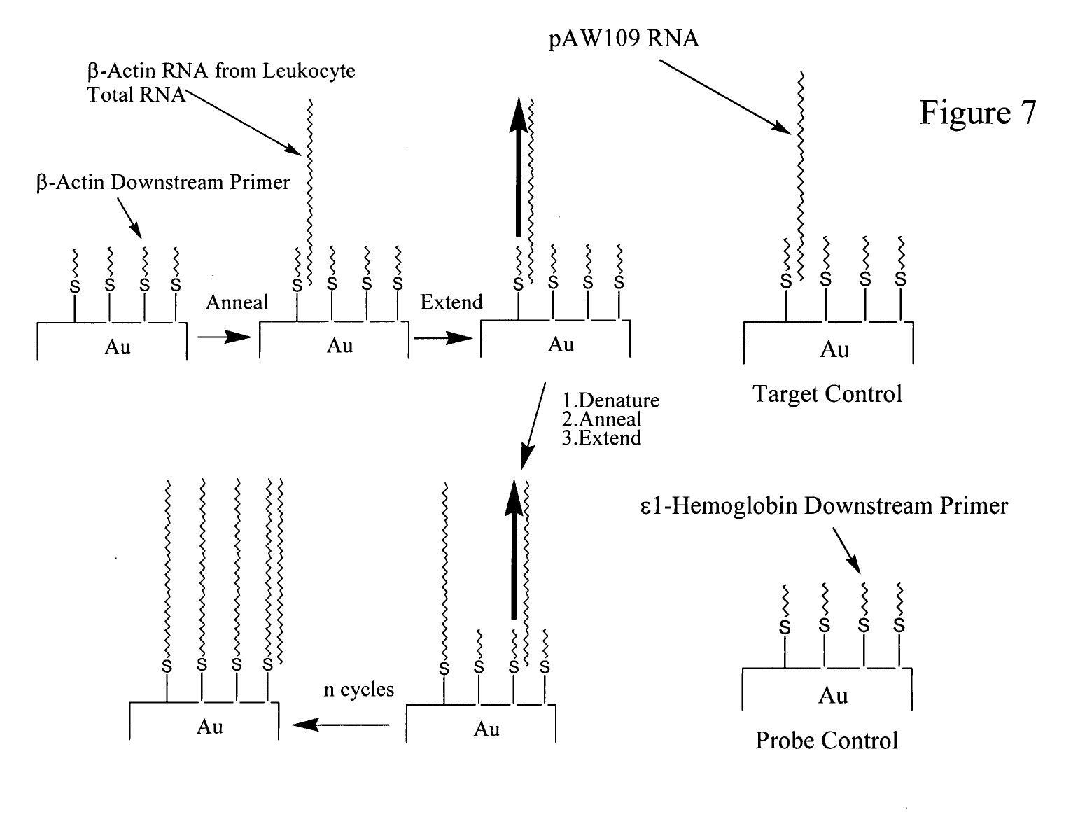

On-electrode Reverse Transcription. BAS 25 μm Au electrodes were cleaned with piranha solution and rinsed with ddH₂O. The electrodes were then derivatized with a 5′-thiolated primer/probe and mercaptohexanol as described. The on-electrode amplification (OERT) solution consisted of 63 μl ddH₂O, 10 μl 10× rTth Reverse Transcriptase Buffer, 10 μl 10 mM MnCl_{2,} 2 μl of α-³²P-dATP, 2 μl each of dCTP (10 mM), dTTP (10 mM), and 7-deaza-dGTP (10 mM), 2 μl rTth thermostable DNA polymerase (2.5 U/μl), 5 μl RNA solution and 1 μl RNAsein RNAse inhibitor. The experimental electrode was derivatized with 1, a downstream primer complementary to the 3′ untranslated region of β-actin mRNA, and the experimental OERT solution contained 5 μl human leukocyte total RNA. The target control electrode was derivatized with 1, and the target control OERT solution contained 5 μl pAW109 plasmid RNA from the rTth DNA polymerase kit. The probe control electrode was derivatized with 2, a downstream primer complementary to the 3′ untranslated region of the human ε1-embryonic hemoglobin mRNA, and the probe control OERT solution contained 5 μl human leukocyte total RNA. The OERT, shown in FIG. 7, consisted of the following protocol: an initial denaturation step of 1 min at 95° C., followed by 30 cycles of 95° C. for 30 sec., 60° C. for 30 sec., extending at 70° C. for 1 min., followed by a final extension at 70° C. for 7 min. Geiger counter readings and cyclic voltammograms were collected after 0 and 30 cycles. The electrode was rinsed thoroughly with R-BFR before Geiger readings and CV's were collected. The experimental reaction and target and probe control reactions were also quantified by using nonradioactive dATP and hybridizing a 5′-γ-³²P-radiolabeled oligonucleotide (probe 3 for β-actin and probe 4 for ε1-embryonic hemoglobin) complementary to the 1^{st} 25 bases, which would be added to the appropriate probe/primer by reverse transcription.

C. Radiochemical Results.

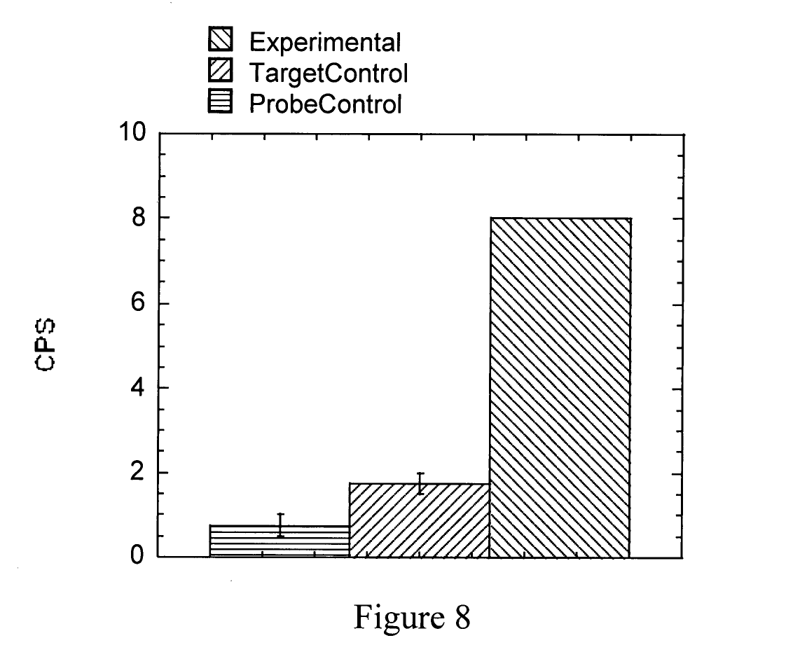

The experimental on-electrode amplification was carried out using human leukocyte total RNA and 1 as the probe/primer. The target control and probe control experiments were carried out in parallel with the experimental OERT protocol as described above. Following 30 cycles of OERT, the electrodes were exposed to the appropriate expression tag, 3 for β-actin and 4 for ε1-hemoglobin. The increases in radioactivity for the experimental, probe control, and target control electrodes are shown in FIG. 8. The data represents the average radioactivities for two separate experiments. The experimental electrode is almost five times as radioactive as the target control electrode and more than ten times as radioactive as the probe control electrode. The small amount of radioactivity at the control electrodes is probably a result of nonspecific adsorption of the post-synthesis tags to the electrode surface. These data indicate that reverse transcription only occurs when the correct target and the complementary probe/primer are present.

We also carried out parallel experimental syntheses using human leukocyte total RNA and the P-actin downstream primer/probe 1 and α-³²P-dATP as an internal label or γ-³²P-labelled post-synthesis tag as an external label. After 30 cycles the α-labeled electrode reads 35 CPS on the Geiger counter, while the γ-labeled electrode reads 2-3 CPS. We calculate that approximately 50 attomoles of α-32 P-dATP have been incorporated on one electrode, while 1.7 to 2.55 attomoles of DNA strands have been synthesized on the other electrode. Thus, we can conclude that each strand has 20-30 A's added during the OERT, and weighting this number against the proportion of G's, T's and C's to A's in the M-actin sequence, we also conclude that about 97-146 bases are added to each strand. We also know that the amount of β-actin mRNA added to each OERT reaction is approximately 0.817 attomoles (W. Lim et al., Veterinary Immunology and Immunopathology 2003, 91, 45-51.; A. Wang et al., PNAS 1989, 86, 9717-9721.; R. de Waal Malefyt et al., J. Exp. Med. 1991, 174, 1209-1220.; Z. Toossi et al., Infect. Immun. 1995, 63, 224-228.; W. Zhong et al., Arch. Surg. 1993, 128, 158-164). Thus, on average each β-actin mRNA molecule acts as a template for the extension of 2-3 probe strands.

D. Electrochemical Results

The experimental on-electrode amplification was carried out using human leukocyte total RNA and 1 as the probe/primer. The target control and probe control experiments were carried out in parallel with the experimental OERT protocol as described above. Incorporation of 7-deazaguanine into the amplified nucleic acid dictates that the redox active metal complex needs to have a potential of approximately 1.0 V to be able to electrochemically detect the modified base. Ru(dmbpy)₃ was chosen as the preferred redox active metal complex. Under the experimental conditions described, electron transfer between the metal complex. Ru(dmbpy)₃ and 7-deazaguanine occurs between 1.0-1.2 V vs. Ag/AgCl reference. Oxidative desorption of the thiol monolayer immobilized on the gold electrode also occurs between 1.0 and 1.2 V. Radiochemical data described above strongly suggest that the on electrode amplification using the RNA targets and the modified nucleobase 7-deazaguanine is occurring, but the loss of the immobilized nucleic acid during the electrochemical interrogation make it difficult to discriminate the electrocatalytic signal of the nucleic acid from the signal due to oxidative desorption of the thiol. Reductive desorption experiments had shown that one electrochemical cycle from 0 to 1.2 V and back, removes a significant fraction of the monolayer. The perceived appearance of specific signal is most likely a result of differing amounts of adsorbed thiol monolayer on the individual electrodes or an interplay between the amplified nucleic acid and the gold electrode which is not well understood.

### EXAMPLE 4

On-Electrode Primer Extension

A. Materials and Methods.

All solutions were made with deionized water (18 mΩcm resistivity) from a Millipore MilliQ system. DNA oligonucleotides were purchased from the UNC-Chapel Hill Lineberger Comprehensive Cancer Center Nucleic Acids Core Facility (Chapel Hill, N.C.). The rTth themostable DNA polymerase kit was purchased from Applied Biosystems (Foster City, Calif.). The Taq themostable DNA polymerase kit was purchased from Promega (Madison, Wis.). Human leukocyte total RNA was isolated from whole blood using the PAXGENE blood RNA validation kit, purchased from Qiagen (Valencia, Calif.). BT474 and HT29 cancer cells were obtained from the laboratory of William Cance, Md. SUM102 cancer cells were obtained from the laboratory of Carolyn Sartor, Md. Total RNA was isolated from frozen cancer cells using the SV total RNA isolation system, purchased from Promega (Madison, Wis.). The upstream and downstream primers used in RTPCR of the β-actin, vitamin-D receptor (VDR), HER2/neu, and Cyclooxygenase type-2 (COX-2) genes are listed in Table 4. N-SH indicates a 5-(CH)₆-thiol modifier, N-PCB indicates a 5′-Photocleavable biotin modifier. US indicates the upstream (5′) primer while DS indicates the downstream (3′) primer. The buffers used were the same as those used by Steel et al. (Anal. Chem. 1998 70, 46704677). DNA deposition was carried out in D-BFR, 1.0 M potassium phosphate buffer, pH 7; electrode rinsing was carried out in R-BFR, 10 mM NaCl, 5 mM Tris buffer, pH 7.4; electrochemical experiments were carried out in 50 mM sodium phosphate buffer, pH 7.4 for cyclic voltammetry and E-BFR, 10 mM Tris buffer, pH 7.4 for chronocoulometry.

Electrode Preparation. Gold macroelectrodes (1.6 mm diameter) and microelectrodes (25 μm diameter) were purchased from Bioanalytical Systems. Before each experiment electrodes were cleaned by immersion in warm piranha solution (70% concentrated sulfuric acid, 30% hydrogen peroxide solution (30% in H₂O)) for 15 min followed by 100% ethanol for 10 min. The electrodes were then rinsed thoroughly with deionized water and immersed in the DNA deposition solutions while still wet.

The modified electrodes were prepared using the procedure of Steel et al. (supra). The clean gold electrode was immersed in a 1.0 μM solution of probe oligonucleotide (DS1-SH, DS2-SH, DS3-SH, or DS4-SH; see Table 4) in D-BFR for 1 h, rinsed with R-BFR for 5 s, immersed in a 1.0 mM 6-mercapto-1-hexanol solution (MCH) solution in deionized water for 0.5 h, and rinsed for 5 s with R-BFR.

Experiments. Cyclic voltammetry (CV) was performed with a BAS100B electrochemical analyzer with a single-compartment voltammetric cell equipped with a 1.6 mm diameter gold pencil-type macroelectrode or a 25 μm diameter gold wire microelectrode as the working electrode, a Pt-wire counter electrode, and a Ag/AgCl reference electrode. The Pt-wire and Ag/AgCl electrodes were purchased from BAS, Inc. Cyclic voltammetry was performed in 50 mM sodium phosphate buffer, pH 7.4, with 100 μM Os(bpy)₃²⁺. For solutions containing Os(bpy)₃²⁺, cyclic voltammograms were taken from 0 to 0.8 V at a scan rate of 100 mV/s. The amount of DNA on the electrodes was determined using chronocoulometry by the method of Steel et al. (supra). Chronocoulommetry was performed in 10 mM Tris buffer, pH 7.4, with 100 μM Ru(NH₃)₆³⁺/100 μM Fe(CN)₆³⁻. All experiments were out at laboratory ambient temperature (21-25° C.).

TABLE 4
Oligonucleotide Sequences.

DS1 (Downstream β-actin primer, U. Michel et al.,
Analytical Biochemistry 1997, 249, 246-247):
5′-AATGTCACGCACGATTTCCC-3′
US1 (Upstream β-actin primer, U. Michel et al.,
Analytical Biochemistry 1997, 249, 246-247):
5′-GATGACCCAGATCATGTTTGAGAC-3′
DS2 (Downstream VDR primer):
5′-CAAACACTTCGAGCACAAGGGGCGTT-3′
US2 (Upstream VDR primer):
5′-CCTGGAGCTGATTGAGCCCCTCATCA-3′
DS3 (HER2/neu Lower primer 2735L, Thomas, et al,
Clinical Cancer Research March 2002, 8, 788-793):
5′-GTAATTTTGACATGGTTGGGACTCTT-3′
US3 (HER2/neu Upper primer 2612U, Thomas, et al,
Clinical Cancer Research March 2002, 8, 788-793):
5′-ACCTGCTGAACTGGTGTATGCA-3′
DS2 (Downstream COX-2 primer):
5′-ATCTTCTTAAGAGGAGCTAAATAGCA-3′
US2 (Upstream COX-2 primer):
5′-TTAGCAGTCCATATCACATTGCAAAA-3′

B. Experimental

On-electrode Primer Extension. BAS 25 μm or 1.6 mm Au electrodes were cleaned with piranha solution/100% ethanol and rinsed with ddH₂O. The electrodes were then derivatized with a 5′-thiolated primer/probe and mercaptohexanol as described. Cyclic voltammograms and chronocoulomograms were then collected with each electrode. RTPCR reactions were carried out using the Applied Biosystems GeneAmp Thermostable reverse transcriptase protocol. Twelve RTPCR reactions were set up using the appropriate primers for each gene (β-actin, VDR, Her-2, or COX-2) and 600 ng total RNA isolated from each cell line (Leukocytes, HT29, and SUM102). Each reaction was performed in triplicate. The upstream primers used were the 5′-PCB derivatives. The RTPCR reactions were carried out using the rTth protocol and the three reactions of each gene/cell line combination were then combined and ethanol precipitated. The combined reactions were then analyzed by agarose gel electrophoresis in the presence of ethidium bromide. Using Roche magnetic streptavidin beads and denaturing the DNA using 1M NaOH we then isolated the sense strand from each reaction. The photocleavable biotin was then photolyzed and the free sense strand was dissolved in 1 mL H-BFR. Prior to on-electrode primer extension the gold electrode derivatized with the appropriate thiolated downstream primer/probe was immersed in the appropriate sense strand/H-BFR solution for 1 hour at 25° C., then rinsed with water. The on-electrode primer extension (OEPE) solution consisted of 253.5 μl ddh₂O, 30 μl 10×PCR Buffer minus Mg²⁺, 9 μl 50 mM MnCl_{2.}2 μl each of 8-oxo-dGTP (10 mM), dCTP (10 mM), dTTP (10 mM), and dATP (10 mM), 1 μl Taq DNA polymerase (5 U/μl). For microelectrodes the volumes of water, buffer and MgCl₂ solution were reduced by two-thirds. Each electrode was then immersed in the OEPE solution and incubated at 72° C for 20 min. The electrodes were then rinsed thoroughly and cyclic voltammograms and chronocoulomograms were again collected.

C. Electrochemical Results

The on-electrode primer extension was carried out using the isolated sense-strands from the each of the twelve RTPCR gene/cell line combinations. Our hypothesis was that if a given cell line expresses a gene of interest, the RTPCR reaction will amplify that mRNA resulting in many “sense-strand” copies of the original mRNA present in the cell line that can be easily isolated from the complementary cDNA transcribed by the reverse transcriptase activity of rTth using the biotin tag on the upstream primers. Since these sense strand copies of the original mRNA inherently have a sequence at their 3′ end which is complementary to the downstream RTPCR primer for the same gene, a 5′-thiolated version of the downstream primer can be used to carry out primer extension on a gold electrode using the sense-strand copy of the mRNA of interest as a template. This primer extension could use Taq polymerase to incorporate 8-oxo-dGTP into the downstream primer/probe, which can then be detected using cyclic voltammetry in the presence of Os(bpy)₃²⁺. RTPCR reactions using cell lines or biological samples that do not express the mRNA of interest will not produce the sense strand copies of the mRNA, and the strepavidin protocol will isolate only the unreacted PCB-modified upstream primers. Since these unextended primers do not contain the sequence complementary to the downstream primer, they will not hybridize to the thiolated downstream primer and cannot be used as a template to incorporate 8-oxo-dGTP during primer extension. Thus, the sense strands isolated using the streptavidin protocol do not need to be further purified, as only cell lines or biological samples which are positive for the mRNA of interest will produce extended upstream primers, and only extended upstream primers will hybridize to the thiolated downstream primer and act as templates for primer extension.

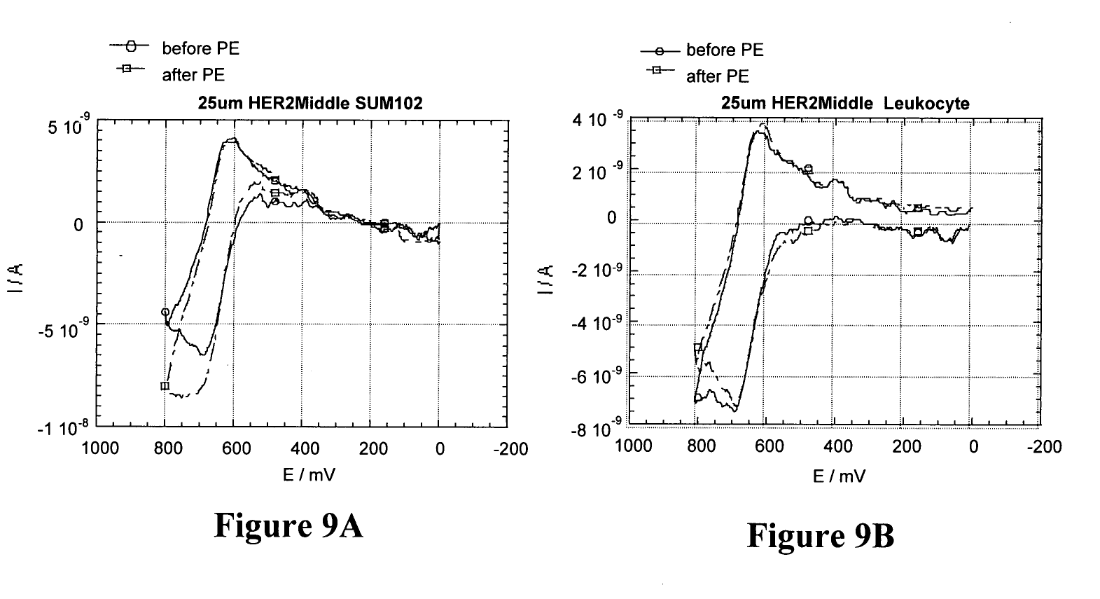

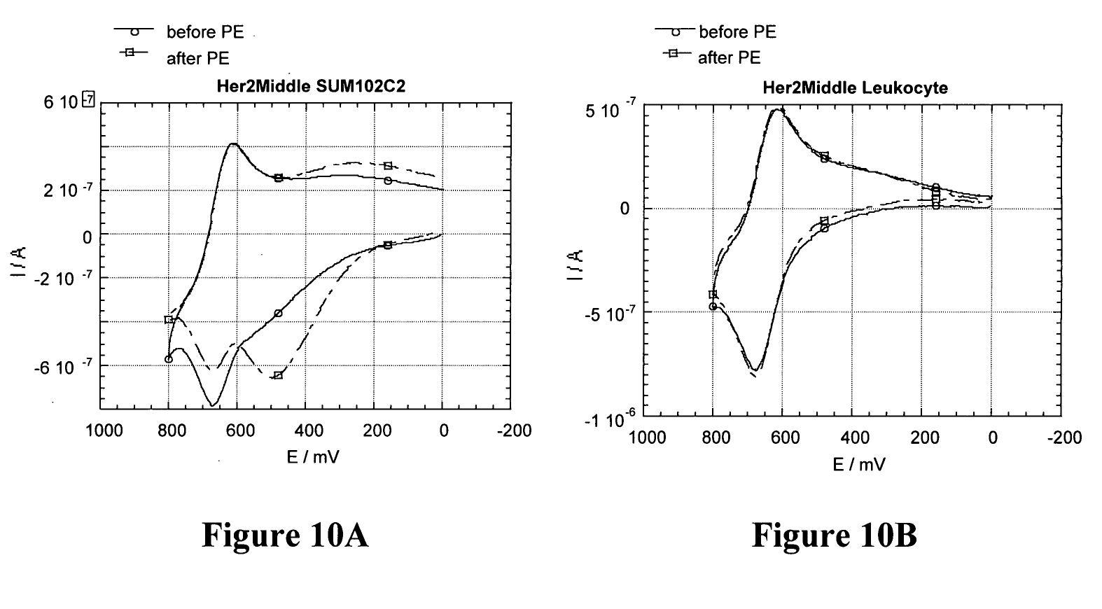

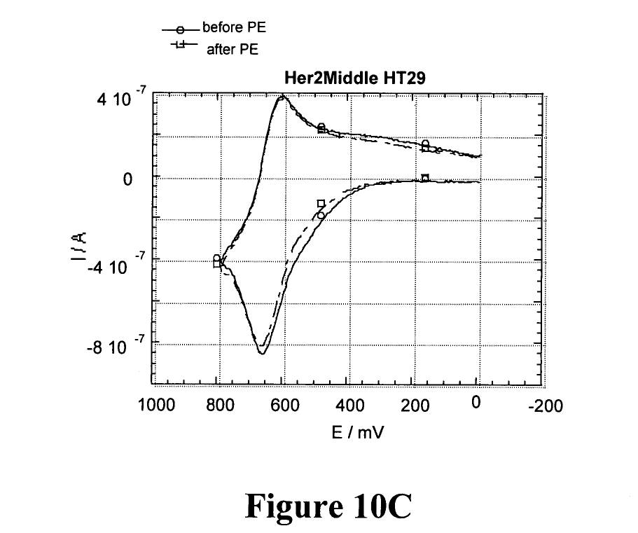

For example, FIG. 9 shows the cyclic voltammograms obtained before and after on-electrode primer extension using DS3-SH-modified 25 μm Au electrodes exposed to the sense strands isolated from the leukocyte and SUM102 Her-2 RTPCR reactions. The electrode exposed to the SUM102 RTPCR product shows noticeable current enhancement due to the catalytic oxidation by Os(bpy)₃²⁺ of the 8-oxo-guanine that has been incorporated into DS3-SH during primer extension. In contrast, the electrode exposed to the leukocyte/Her-2 RTPCR reaction shows no such current enhancement because there are only unextended upstream primers present that are unable to hybridize to DS3-SH. FIG. 10 shows the same experiment as FIG. 9, but using macroelectrodes. Again, there is current enhancement in the CV using the electrode exposed to the SUM102/Her-2 RTPCR reaction, while the electrodes exposed to the HT29/Her-2 or leukocyte/Her-2 reactions show no current enhancement. These experiments were carried out using all of the cell-line/gene combinations, and current enhancement was observed only at electrodes exposed to RTPCR reactions from cell-lines positive for the mRNA of interest. In the case of COX-2, current enhancement was observed only at electrodes exposed to the leukocyte/COX-2 RTPCR reaction, while electrodes exposed to the SUM102/COX-2 and HT29/COX-2 RTPCR reactions produced no catalytic current. With β-actin and VDR, however, catalytic current enhancement was observed for electrodes exposed to all three cell-line/gene combinations. This result is consistent with the fact that separate RTPCR experiments confirmed that both genes are expressed in all three cell lines.

### EXAMPLE 5

Catalytic Chronocoulometry-Coupled On-Electrode Reverse Transcription

A. Materials and Methods.

All solutions were made with deionized water (18 mΩcm resistivity) from a Millipore MilliQ system. DNA oligonucleotides were purchased from the UNC-Chapel Hill Lineberger Comprehensive Cancer Center Nucleic Acids Core Facility (Chapel Hill, N.C.). The rTth themostable DNA polymerase kit was purchased from Applied Biosystems (Foster City, Calif.). The Taq themostable DNA polymerase kit was purchased from Promega (Madison, Wis.). Human leukocyte total RNA was isolated from whole blood using the PAXGENE blood RNA validation kit, purchased from Qiagen (Valencia, Calif.). BT474 and HT29 cancer cells were obtained from the laboratory of William Cance, Md. SUM102 cancer cells were obtained from the laboratory of Carolyn Sartor, Md. Total RNA was isolated from frozen cancer cells using the SV total RNA isolation system, purchased from Promega (Madison, Wis.). The upstream and downstream primers used in RTPCR of the β-actin, vitamin-D receptor (VDR), HER2/neu, and Cyclooxygenase type-2 (COX-2) genes are listed in Table 5. N-SH indicates a 5-(CH)₆-thiol modifier, N-PCB indicates a 5′-Photocleavable biotin modifier. US indicates the upstream (5′) primer while DS indicates the downstream (3′) primer, PS indicates the post-synthesis tag, a sequence that is complementary to the cDNA synthesized as the mRNA is reverse transcribed on the electrode. The buffers used were the same as those used by Steel et al. (Anal. Chem. 1998 70, 4670-4677). DNA deposition was carried out in D-BFR, 1.0 M potassium phosphate buffer, pH 7; electrode rinsing was carried out in R-BFR, 10 mM NaCl, 5 mM Tris buffer, pH 7.4; electrochemical experiments were carried out in 50 mM sodium phosphate buffer, pH 7.4 for cyclic voltammetry and E-BFR, 10 mM Tris buffer, pH 7.4 for chronocoulometry.

Electrode Preparation. Gold microelectrodes (25 μm diameter) were purchased from Bioanalytical Systems. Before each experiment electrodes were cleaned by immersion in warm piranha solution (70% concentrated sulfuric acid, 30% hydrogen peroxide solution (30% in H₂O)) for 15 min followed by 100% ethanol for 10 min. The electrodes were then rinsed thoroughly with deionized water and immersed in the DNA deposition solutions while still wet.

The modified electrodes were prepared using the procedure of Steel et al. (supra). The clean gold electrode was immersed in a 1.0 μM solution of probe oligonucleotide (DSI-SH, DS2-SH, DS3-SH, or DS4-SH) in D-BFR for 1 h, rinsed with R-BFR for 5 s, immersed in a 1.0 mM 6-mercapto-1-hexanol solution (MCH) solution in deionized water for 0.5 h, and rinsed for 5 s with R-BFR.

Experiments. Chronocoulommetry (CC) was performed with a BAS100B electrochemical analyzer with a single-compartment voltammetric cell equipped with a 25 μm diameter gold wire microelectrode as the working electrode, a Pt-wire counter electrode, and a Ag/AgCl reference electrode. The Pt-wire and Ag/AgCl electrodes were purchased from BAS, Inc. The amount of DNA on the electrodes was determined using the method of Steel et al. (supra). Chronocoulommetry was performed in 10 mM Tris buffer, pH 7.4, with 100 μM Ru(NH₃)₆³⁺/100 μM Fe(CN)₆³⁻. (M. Lapierre et. al., Anal. Chem. 75 (2003) 6327) All experiments were carried out at laboratory ambient temperature (21-25° C.).

TABLE 5
Oligonucleotide Sequences

DS1 (Downstream β-actin primer, U. Michel et al.,
Analytical Biochemistry 1997, 249, 246-247):
5′-AATGTCACGCACGATTTCCC-3′
US1 (Upstream β-actin primer, U. Michel et al.,
Analytical Biochemistry 1997, 249, 246-247):
5′-GATGACCCAGATCATGTTTGAGAC-3′
PS1 (β-actin post-synthesis tag):
5′-TGTCGAAGTGGTGGTGCCGGCTCG-3′
DS2 (Downstream VDR primer):
5′-CAAACACTTCGAGCACAAGGGGCGTT-3′
US2 (Upstream VDR primer):
5′-CCTGGAGCTGATTGAGCCCCTCATCA-3′
PS2 (VDR Post-synthesis Tag):
5′-TCCAGCCTGAGTGCAGCATGAAGCTA-3′
DS3 (HER2/neu Lower primer 2735L, Thomas, et al,
Clinical Cancer Research March 2002, 8, 788-793):
5′-GTAATTTTGACATGGTTGGGACTCTT-3′
US3 (HER2/neu Upper primer 2612U, Thomas, et al,
Clinical Cancer Research March 2002, 8, 788-793):
5′-ACCTGCTGAACTGGTGTATGCA-3′
PS3 (HER2/neu Post-synthesis Tag):
5′-TGCATACACCAGTTCAGCAGGT-3′
DS4 (Downstream COX-2 primer):
5′-ATCTTCTTAAGAGGAGCTAAATAGCA-3′
US4 (Upstream COX-2 primer):
5′-TTAGCAGTCCATATCACATTGCAAAA-3′
PS4 (COX-2 Post-synthesis Tag):
5-TTCAGGTAAACCTCAGCTCAGGACTG-3′

B. Experimental.

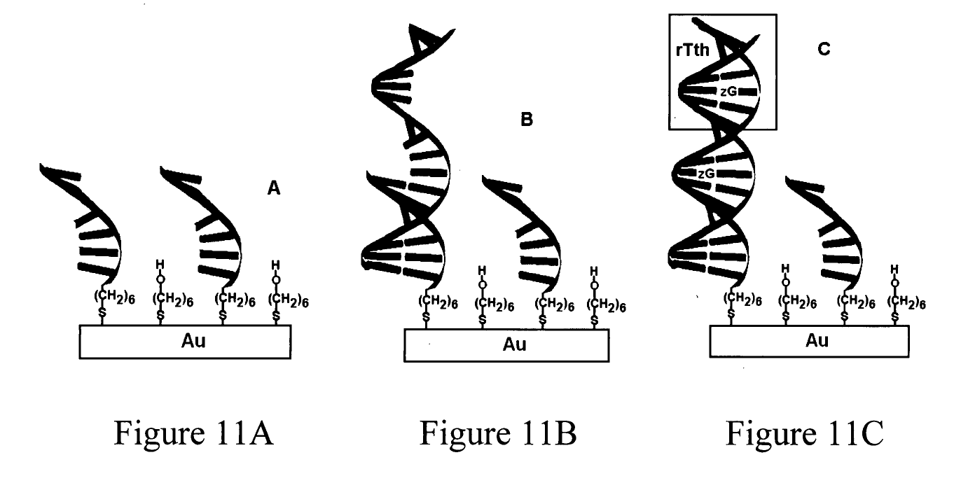

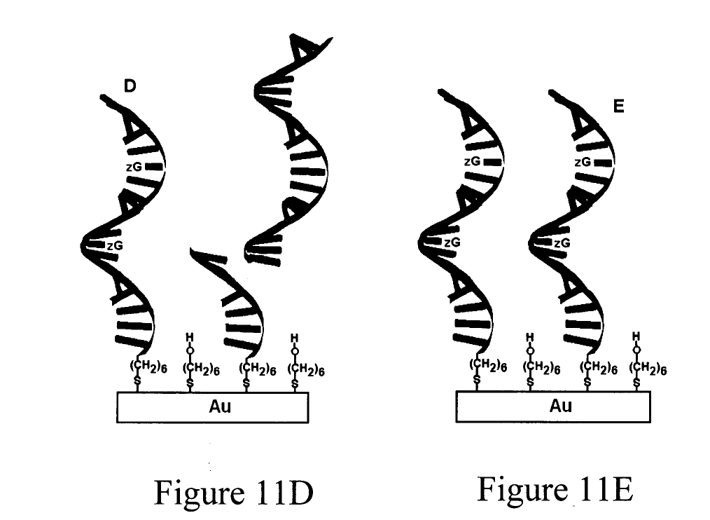

Catalytic Chronocoulometry-Coupled On-Electrode Reverse Transcription. BAS 25 μm Au electrodes were cleaned with piranha solution/100% ethanol and rinsed with ddH₂O. The electrodes were then derivatized with a 5′-thiolated primer/probe and mercaptohexanol as described. Chronocoulomograms were then collected with each electrode. On-Electrode Reverse Transcription (OERT) reactions were carried out using a modification of the Applied Biosystems GeneAmp Thermostable reverse transcriptase protocol. The OERT solution consisted of 70 μl RNAse-free ddH₂O, 10 μl 10× rTth Reverse Transcriptase Buffer, 10 μl 10 mM MnCl_{2.}2 μl each of dATP(10 mM), dCTP (10 mM), dTTP (10 mM), and dGTP (10 mM), 2 μl rTth thermostable DNA polymerase (2.5 U/μl), 600 ng total RNA and 0.1 μl RNAsein RNAse inhibitor (Promega, Madison, Wis.). Each derivatised electrode was then immersed in the OERT solution and incubated at 37° C. for 20 min followed by 70° C. for 20 min. The electrodes were then subjected to 30 OERT cycles. The OERT, shown in FIG. 11, consisted of the following protocol: an initial denaturation step of 30 sec. at 90° C., followed by 30 cycles of 90° C. for 10 sec., 60° C. for 30 sec., extending at 70° C. for 1 min., followed by a at 70° C. for 7 min. The mRNA was then denatured from the probe by heating to 90° C. for 10 sec. Geiger counter readings and chronocoulommograms were collected after 0 and 30 cycles. The electrode was rinsed thoroughly with R-BFR before Geiger readings and CC's were collected. Following OERT, the electrodes were immersed in a 1 μM solution of the appropriate post-synthesis tag at 37° C. for 1 hour. The electrodes were then rinsed thoroughly and chronocoulommograms were again collected. The experimental on-electrode reverse transcription was carried out using 600 ng BT474, HT29 and Leukocyte total RNA and thiolated probe/primer oligonucleutides for the Her2/neu, COX-2, β-actin, and VDR genes. Table 6 shows the number of moles and molecules of each gene of interest in 600 ng total RNA from a given cell line determined by multiplex relative RTPCR. An omitted gene/cell line combination indicates that expression of the gene in that cell line is not detectable by RTPCR.

C. Electrochemical results.

We used a modification of the methods used by Tarlov and coworkers and Kelly and coworkers to monitor the mRNA-directed synthesis of cDNA on the electrode surface. By collecting chronocoulomograms in E-BFR containing both 100 μm Ru(NH₃)₆³⁺ and 100 μm Fe(CN)₆³⁻, we can determine the surface density and length of the probe oligonucleotide and the density of hybridized mRNA while simultaneously detecting a catalytic increase in charge resulting from the reduction of Fe(CN)₆³⁻ by Ru(NH₃)₆³⁺. This results from the fact that the integrated Cottrell equation, equation 1,
Q=(2nFAD₀^{1/2}C₀/π^{1/2})t^{1/2}+Q_{dl}+nFAΓ₀ 1
Γ_{DNA}=Γ₀(z/m)N_{A} 2
contains terms which describe both surface and solution electrochemical processes. Fe(CN)₆³⁻ is repelled by the negatively charged DNA film on the electrode, and it is confined to the bulk solution. Thus, the catalytic reduction of Fe(CN)₆³⁻ by Ru(NH₃)₆²⁻ is described only by the first term of the Cottrell equation, which describes solution processes. We should be able to measure a catalytic increase in collected charge at the end of the chronocoulometric pulse that results from the shuttling of electrons from DNA-bound Ru(NH₃)₆³⁺ which is reduced to Ru(NH₃)₆²⁺ and subsequently reduces Fe(CN)₆³⁻ by one electron. This catalytic process should be amplified by an increase in the amount of DNA at the surface, e.g. when the surface-bound probes are extended using the mRNA of interest as a template. In the absence of the mRNA of interest, the amount of charge collected before and after OERT should remain essentially constant.

The direct reduction of Ru(NH₃)₆³⁺ bound to the phosphate backbone of the DNA on the electrode surface is essentially instantaneous, and therefore described by the last term of the Cottrell equation, the surface term. Coupled with equation 2, this term allows us to calculate the surface density of DNA on the electrode (Γ_{DNA}) before OERT. Following OERT, if we assume that the DNA surface density has remained essentially the same, we can now determine the length of the probe oligonucleotides on the electrode surface. If the mRNA of interest is not present in the experiment, the length of the probes (m) should remain unchanged, since no extension of these probes will have taken place. In contrast, if the mRNA of interest is present, the thermostable reverse transcriptase should have added many bases to the probe, which should be detectable using equation 2 and the surface term of equation 1. Finally, we can determine roughly how many of the probes have been extended, if any, by hybridizing the post-synthesis tag to the extended probe strands and measuring the new Γ_{DNA} chronocoulommetrically.

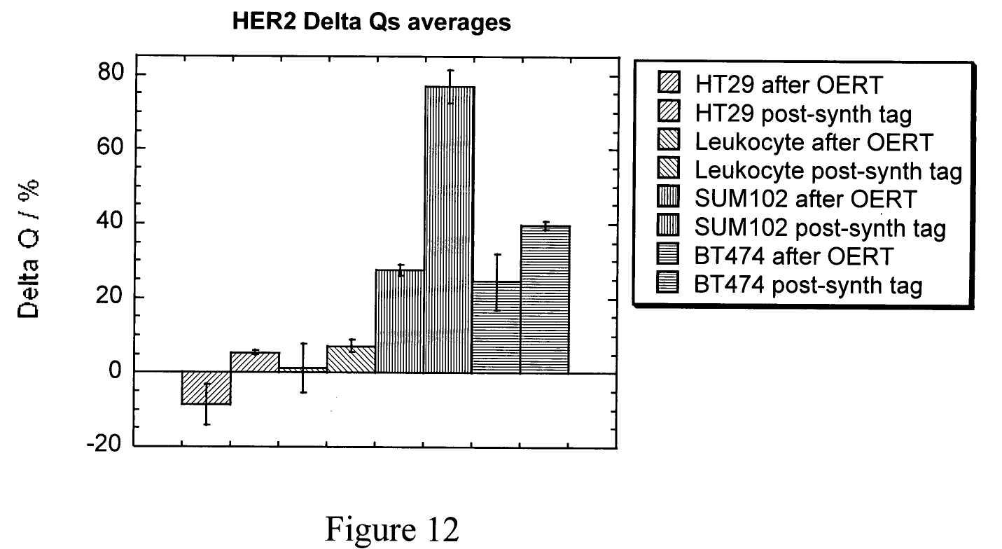

FIG. 12 shows the percent change in the amount of charge collected (ΔQ) before and after OERT and following hybridization of the post-synthesis tag when using DS3-SH as the surface probe. Since the Her-2 mRNA is expressed only in the SUM102 and BT474 breast cancer cell lines, a large catalytic increase in collected charge is observed when these total RNA samples are used in the OERT reaction, while the amount of charge collected when HT29 or leukocyte total RNA are used remains essentially unchanged.

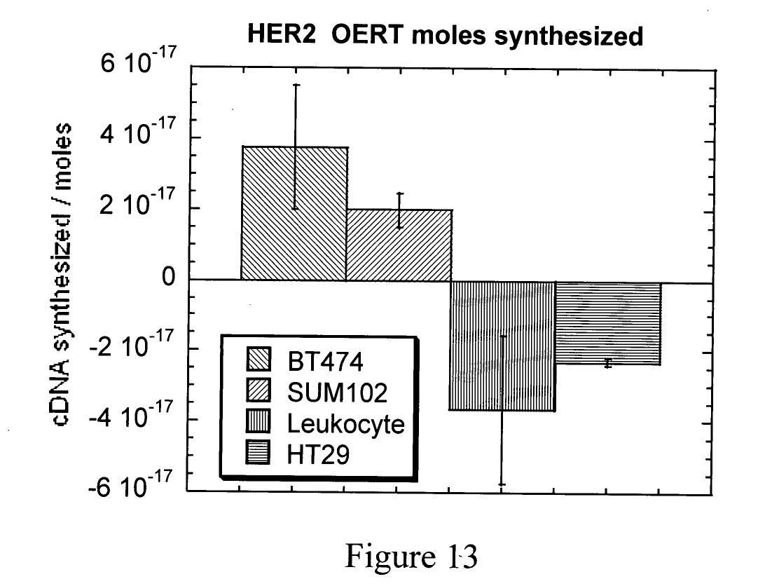

FIG. 13 shows the number of moles of cDNA synthesized on the electrode surface during OERT using DS3-SH as the surface probe. The use of SUM102 or BT474 total RNA samples results in the extension of 20-30 attomoles of probe strands on the electrode surface, while HT29 and leukocyte total RNA samples, which contain no Her-2 mRNA, do not result in the synthesis of any cDNA.

TABLE 6
Multiplexed RTPCR Results

| | | Moles/600 ng | Molecules/ |
|---|---|---|---|
| Gene | Cell Line | Total RNA | 600 ng |
| β-actin | Leukocytes | 1.05e−16 | 6.3e7 |
| β-Actin | BT474 | 8.43e−16 | 5.1e8 |
| β-Actin | HT29 | 3.76e−18 | 2.3e6 |
| VDR | Leukocytes | 3.14e−18 | 1.9e6 |
| VDR | BT474 | 1.7e−16 | 1.03e8 |
| VDR | HT29 | 2.14e−18 | 1.3e6 |
| Her-2 | SUM102 | 1.22e−18 | 7.5e5 |
| Her-2 | BT474 | 9.5e−19 | 5.7e5 |
| COX-2 | Leukocytes | 2.5e−17 | 1.5e7 |

### EXAMPLE 6

Use of Signal Probe for Detection of RNA and DNA Targets Without On-Electrode Amplification

Gold electrodes modified with an appropriate thiolated capture probe and mercaptohexanol will be exposed to a solution containing a nucleic acid target, either RNA or DNA. The target will hybridize if it is complementary to the capture probe immobilized on the gold electrode surface. A signal probe, complementary to a portion of the hybridized target nucleic acid, which contains 8-oxoguanine or any other suitable preselected base, will then be hybridized to the nucleic acid target. The preselected base will be detected electrochemically using the appropriate metal mediator. If the signal probe contains 8-oxoguanine then Os(bpy)₃²⁺ would be used. If the target does not hybridize, then the signal probe will not hybridize and no catalytic electrochemical signal will be detected.

### EXAMPLE 7

Use of Signal Probe for Detection of RNA and DNA Targets With On-Electrode Amplification

A. Materials and Methods

All solutions were made with deionized water (18 mΩcm resistivity) from a Millipore MilliQ system. DNA oligonucleotides were purchased from the UNC-Chapel Hill Lineberger Comprehensive Cancer Center Nucleic Acids Core Facility (Chapel Hill, N.C.). The rTth themostable DNA polymerase kit was purchased from Applied Biosystems (Foster City, Calif.). The Taq themostable DNA polymerase kit was purchased from Promega (Madison, Wis.). Human leukocyte total RNA was isolated from whole blood using the PAXGENE blood RNA validation kit, purchased from Qiagen (Valencia, Calif.). BT474 and HT29 cancer cells were obtained from the laboratory of William Cance, MD. SUM102 cancer cells were obtained from the laboratory of Carolyn Sartor, Md. Total RNA was isolated from frozen cancer cells using the SV total RNA isolation system, purchased from Promega (Madison, Wis.). Terminal Transferase from calf thymus, CoCl₂, and TdT reaction buffer were purchased from Roche(Mannheim, Germany). 8-oxo-2′-deoxyguanosine triphosphate was purchased from Tri-Link Biotechnologies. The upstream and downstream primers used in RTPCR of the β-actin, vitamin-D receptor (VDR), HER2/neu, and Cyclooxygenase type-2 (COX-2) genes are listed in Table 7. N-SH indicates a 5-(CH)₆-thiol modifier, N-PCB indicates a 5′-Photocleavable biotin modifier. US indicates the upstream (5′) primer while DS indicates the downstream (3′) primer, PS indicates the post-synthesis tag, a sequence that is complementary to the cDNA synthesized as the mRNA is reverse transcribed on the electrode. The buffers used were the same as those used by Steel et al. (Anal. Chem. 1998 70, 4670-4677). DNA deposition was carried out in D-BFR, 1.0 M potassium phosphate buffer, pH 7; electrode rinsing was carried out in R-BFR, 10 mM NaCl, 5 mM Tris buffer, pH 7.4; electrochemical experiments were carried out in 50 mM sodium phosphate buffer, pH 7.4 for cyclic voltammetry and E-BFR, 10 mM Tris buffer, pH 7.4 for chronocoulometry.

Electrode Preparation. Gold microelectrodes (25 μm diameter) were purchased from Bioanalytical Systems. Before each experiment electrodes were cleaned by immersion in warm piranha solution (70% concentrated sulfuric acid, 30% hydrogen peroxide solution (30% in H₂O)) for 15 min followed by 100% ethanol for 10 min. The electrodes were then rinsed thoroughly with deionized water and immersed in the DNA deposition solutions while still wet.

The modified electrodes were prepared using the procedure of Steel et al. (supra). The clean gold electrode was immersed in a 1.0 μM solution of probe oligonucleotide (DS1-SH, DS2-SH, DS3-SH, or DS4-SH) in D-BFR for 1 h, rinsed with R-BFR for 5 s, immersed in a 1.0 mM 6-mercapto-1-hexanol solution (MCH) solution in deionized water for 0.5 h, and rinsed for 5 s with R-BFR.

8-oxo-2′-deoxyguanosine Triphosphate End Labeling. The post-synthesis tags (signal probes) PS1-PS4 were 3′-end-labeled using the Roche TdT 3′ endlabeling reaction protocol. 10 μl 5× reaction buffer, 40 pmol PS1,2,3, or 4, 5 μl 2.5 mM CoCl₂, 10 μl 1 mM 8-oxo-dGTP and 14 μl DNase/RNase-free H₂O were combined with 25 units(1 μl) TdT and incubated at 37° C. for 60 minutes. The labeled DNA was then purified using a spin column and diluted in H-BFR (300 μI for macroelectrodes, 50 μl for microelectrodes).

Experiments. Cyclic Voltammetry (CV) was performed with a BAS100B electrochemical analyzer with a single-compartment voltammetric cell equipped with a 25 μm diameter gold wire microelectrode as the working electrode, a Pt-wire counter electrode, and a Ag/AgCl reference electrode. The Pt-wire and Ag/AgCl electrodes were purchased from BAS, Inc. Cyclic Voltammetry was performed in 100 mM sodium phosphate buffer, pH 7.4, with 100 μM Os(bpy)₃²⁺. All experiments were carried out at laboratory ambient temperature (21-25° C.).

TABLE 7
Oligonucleotide Sequences

DS1 (Downstream β-actin primer, U. Michel et al.,
Analytical Biochemistry 1997, 249, 246-247):
5′-AATGTCACGCACGATTTCCC-3′
US1 (Upstream β-actin primer, U. Michel et al.,
Analytical Biochemistry 1997, 249, 246-247):
5′-GATGACCCAGATCATGTTTGAGAC-3′
PS1 (β-actin post-synthesis tag):
5′-TGTCGAAGTGGTGGTGCCGGCTCG-3′
DS2 (Downstream VDR primer):
5′-CAAACACTTCGAGCACAAGGGGCGTT-3′
US2 (Upstream VDR primer):
5′-CCTGGAGCTGATTGAGCCCCTCATCA-3′
PS2 (VDR Post-synthesis Tag):
5′-TCCAGCCTGAGTGCAGCATGAAGCTA-3′
DS3 (HER2/neu Lower primer 2735L, Thomas, et al,
Clinical Cancer Research March 2002, 8, 788-793):
5′-GTAATTTTGACATGGTTGGGACTCTT-3′
US3 (HER2/neu Upper primer 2612U, Thomas, et al,
Clinical Cancer Research March 2002, 8, 788-793):
5′-ACCTGCTGAACTGGTGTATGCA-3′
PS3 (HER2/neu Post-synthesis Tag):
5′-TGCATACACCAGTTCAGCAGGT-3′
DS4 (Downstream COX-2 primer):
5′-ATCTTCTTAAGAGGAGCTAAATAGCA-3′
US4 (Upstream COX-2 primer):
5′-TTAGCAGTCCATATCACATTGCAAAA-3′
PS4 (COX-2 Post-synthesis Tag):
5′-TTCAGGTAAACCTCAGCTCAGGACTG-3′

B. Experimental

8-oxo-Guanine-Labeled-Signal Probe-Coupled On-Electrode Reverse Transcription. BAS 25 μm Au electrodes were cleaned with piranha solution/100% ethanol and rinsed with ddH₂O. The electrodes were then derivatized with a 5′-thiolated primer/probe and mercaptohexanol as described. Cyclic voltammograms were then collected with each electrode. On-Electrode Reverse Transcription (OERT) reactions were carried out using a modification of the Applied Biosystems GeneAmp Thermostable reverse transcriptase protocol. The OERT solution consisted of 70 μl RNAse-free ddH₂O, 10 μl 10× rTth Reverse Transcriptase Buffer, 10 μl 10 mM MnCl_{2.} 2 μl each of dATP(10 mM), dCTP (10 mM), TTP (10 mM), and dGTP (10 mM), 2 μl rTth thermostable DNA polymerase (2.5 U/μl), 600 ng total RNA and 0.1 μl RNAsein RNAse inhibitor (Promega, Madison, Wis.). Each derivatised electrode was then immersed in the OERT solution and incubated at 37° C. for 60 min followed by 70° C. for 30 min. The mRNA was then denatured from the probe by heating to 90° C. for 10 sec and the electrodes were immersed in a 0.13 μM (macroelectrodes) or 0.8 μM (microelectrodes) solution of the appropriate post-synthesis tag at 37° C. for 1 hour. The electrodes were then rinsed thoroughly and cyclic voltammograms were again collected.

C. Electrochemical Results.

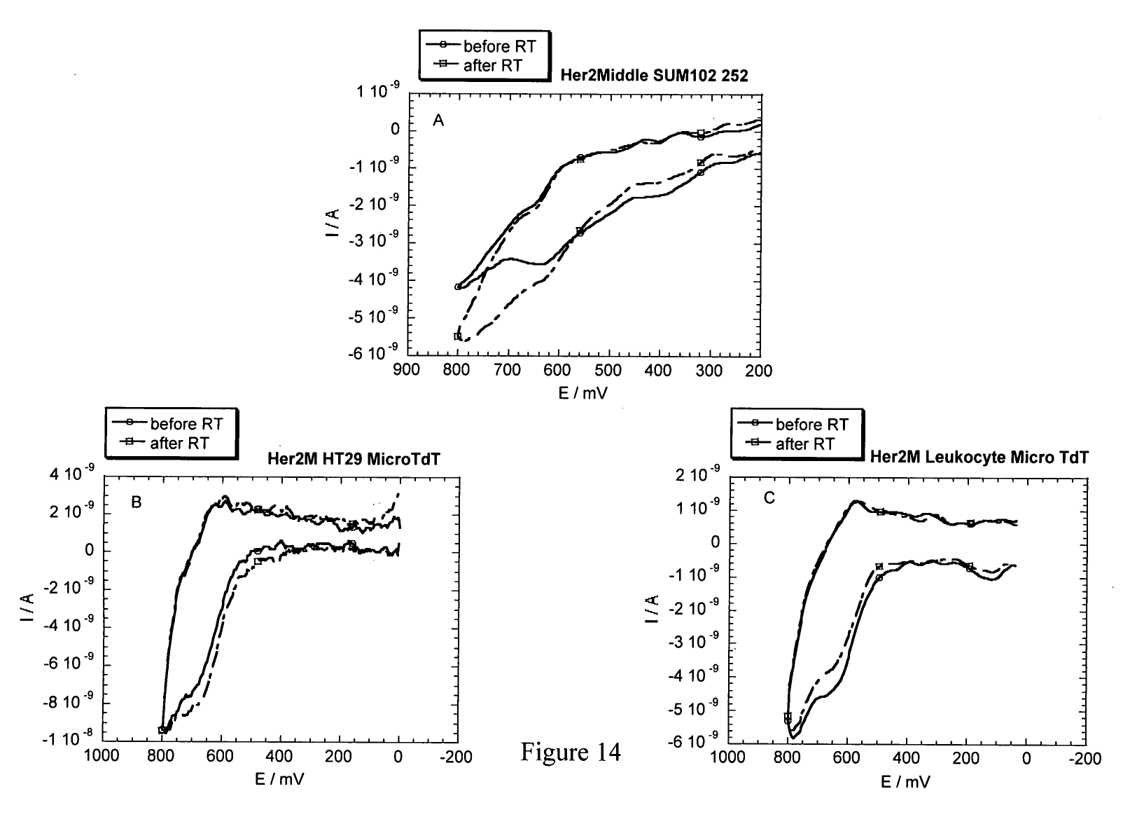

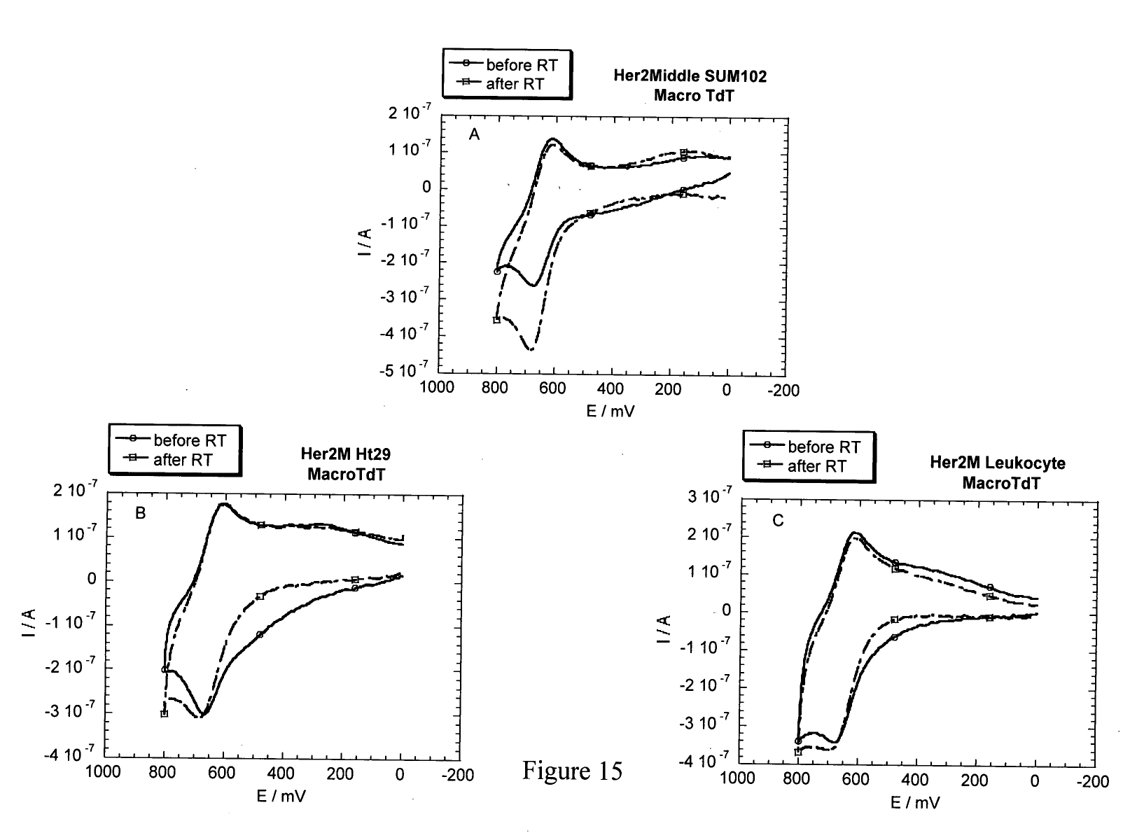

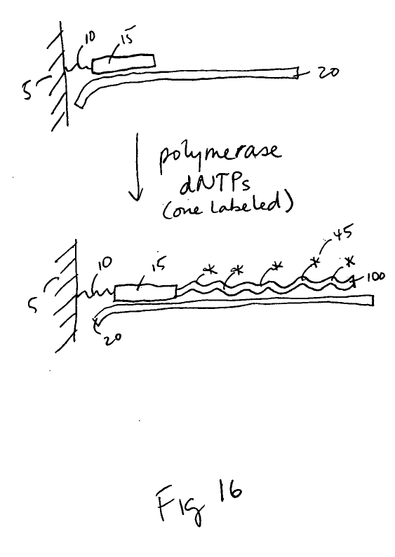

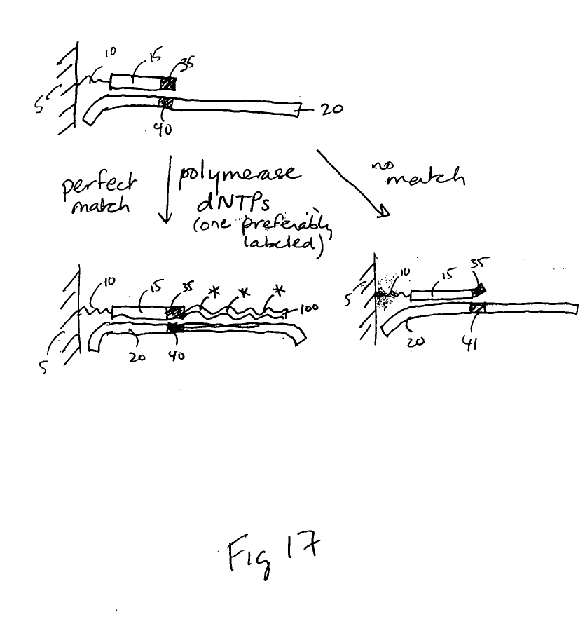

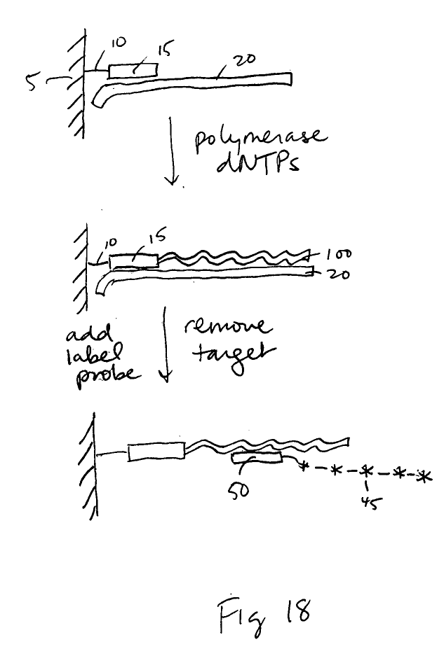

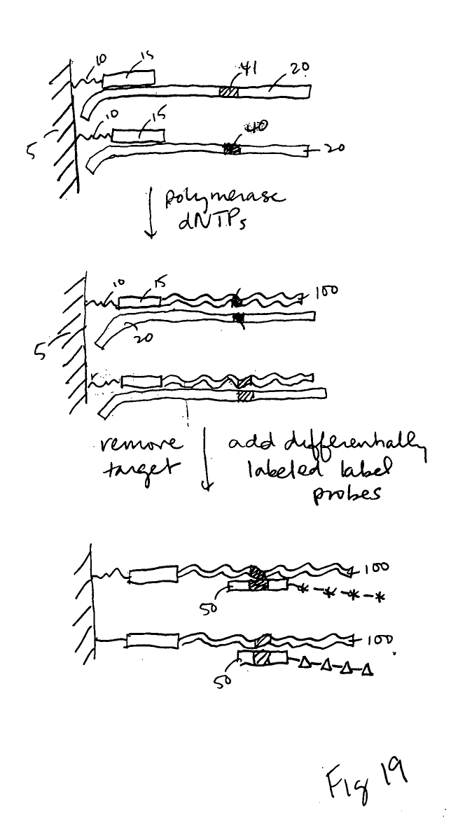

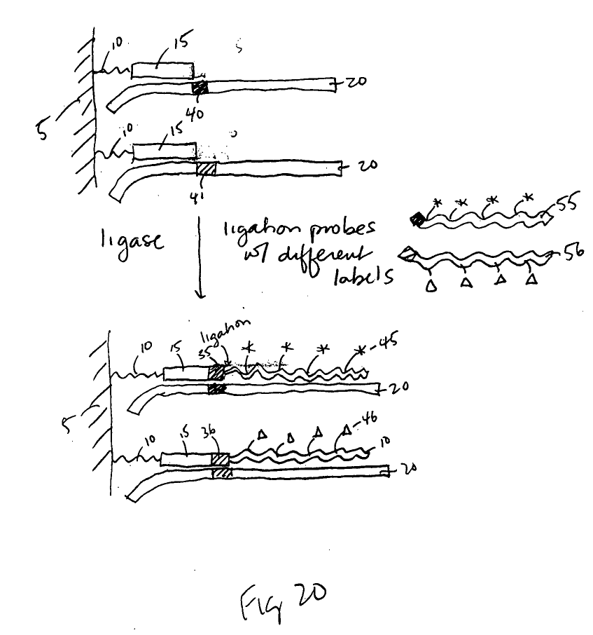

The OERT was carried out as described above using DS3-SH as the probe. Parallel experiments were carried out using either micro- or macro-electrodes and using SUM102, HT29, or Leukocyte total NA as the target. The electrodes were then incubated in PS3 that had been 3′-end-labeled with 8-oxo-dGTP as described above and the CV's were collected in 100 μM Osbpy. FIGS. 14A and 15A show, the DS3-SH-modified electrodes show noticeable catalytic current when OERT is carried out using SUM102 total RNA as the target and the electrodes are incubated in 8-oxo-G labeled-PS3. Conversely, FIGS. 14B and C and 15B and C show no catalytic current after incubation in PS3, indicating that no extended thiolated probes are present to bind the labeled PS3 signal probes.

The experiment described above can also be done using DNA as the target nucleic acid. For the DNA example, the amplification mixture used to extend the primer would contain Taq polymerase, the appropriate buffer and dNTPs. In addition, the amplification mixture could, if desired, contain 8-oxoguanine triphosphate in place of the guanine triphosphate. Electrochemical signal will be generated from the incorporation of the 8-oxoguanine into the extended primer and from the hybridized signal probe.

### EXAMPLE 8

One-Pot Detection of RNA by Extension of a cDNA Product Thereof

A method for the on-electrode primer extension using RNA as a target and 8-oxoguanine as the preselected base will be described. Typical enzymes used to amplify RNA targets do not readily accept 8-oxoguanine. Typical enzymes used to amplify DNA targets do accept 8-oxoguanine. The methodology which will be described entails the synthesis of a small number of strands of cDNA from an RNA target. The cDNA is captured by the probe immobilized on the gold electrode and used as the template for the on-electrode primer extension.

Gold electrodes modified with an appropriate thiolated capture probe and mercaptohexanol are exposed to a solution containing a RNA target, appropriate primer, dNTPs, including 8-oxoguanine triphosphate, transcriptor reverse transcriptase, RNase inhibitor, Taq DNA polymerase and an appropriate reaction buffer. The first step of the reaction is the amplification in solution of the RNA to yield several copies of the cDNA product under appropriate reaction conditions. One of the key elements of the successful implementation of this approach will be to limit the amount of dGTP in the reaction mixture, so that little to no dGTP is available for insertion during the on-electrode primer extension. The cDNA product will be allowed to hybridize to the capture probe immobilized on the electrode surface. Taq polymerase will then be used to do the on-electrode primer extension using the cDNA as a template under the appropriate reaction conditions. 8-oxoguanine will be inserted and used as the preselected base for the electrochemical detection.

### Examples 9

6-mercaptoguanine as a Detectable Base

These examples are carried out in like manner as Examples 2 and 3, but using 6-mercaptoguanine as the detectable or preselected base instead of 8-oxoguanine. 6-mercaptoguanine will be used as the preselected base in the on electrode primer extension for both RNA and DNA targets. Synthesis and purification of the 6-mercaptoguanine triphosphate has been described in RNA 3 (1997) 464467. Experiments have demonstrated that 6-mercaptoguanosine, the 6-mercaptoguanine triphosphate precursor is electrochemically active. Metal mediated oxidation of the 6-mercaptoguanosine using Os(bpy)₃²⁺ yields a current approximately 3-5 times greater than background in the potential range of 0.6-0.8 V (vs. Ag/AgCl reference electrode).

The foregoing examples are illustrative of the present invention, and are not to be construed as limiting thereof. The invention is described by the following claims, with equivalents of the claims to be included therein.

1. A method of detecting at least a first target nucleic acid sequence in a sample comprising:
(a) providing a solid support comprising at least a first gold electrode having a first primer oligonucleotide immobilized thereon;
(b) hybridizing said first target sequence to said first primer oligonucleotide to form a first assay complex;
(c) elongating said first primer oligonucleotide in a reaction mixture with an enzyme and a plurality of preselected first detectable nucleotides to produce a first elongated oligonucleotide;
(d) reacting said first elongated oligonucleotide with a first transition metal complex that oxidizes said detectable nucleotide in a first oxidation-reduction reaction, regenerating the reduced form of said first transition metal complex in a catalytic reaction;
(e) detecting the presence of said first target sequence by detecting said first oxidation-reduction reaction.

2. A method according to claim 1, wherein said sample further comprises a second target sequence, said solid support further comprises a second electrode with a second oligonucleotide immobilized thereon, and said method further comprising:
(a) hybridizing said second target sequence to said second primer oligonucleotide to form a second assay complex;
(c) elongating said second primer oligonucleotide in a reaction mixture with an enzyme and a plurality of preselected second detectable nucleotides to produce a second elongated oligonucleotide;
(d) reacting said second elongated oligonucleotide with a second transition metal complex that oxidizes said detectable nucleotide in a second oxidation-reduction reaction, regenerating the reduced form of said second transition metal complex in a catalytic reaction;
(e) detecting the presence of said second target sequence by detecting said second oxidation-reduction reaction.

3. A method according to claim 2 wherein said first preselected second detectable nucleotide is the same as said preselected second detectable nucleotide.

4. A method according to claim 1 wherein said enzyme is a polymerase and reaction mixture comprises a set of at least four different dNTPs one of which is a preselected detectable nucleotide, such that the elongated oligonucleotide comprises said detectable nucleotides.

5. A method according to claim 1 wherein said enzyme is a ligase and said plurality of preselected detectable nucleotides are contained within a ligation probe, wherein if said ligation probe hybridizes adjacently to said primer oligonucleotide on said target sequence, ligation occurs and a ligation product is formed that comprises said detectable nucleotides.

6. A method according to claim 1 wherein said target molecule is a circular probe, said enzyme is a polymerase and said reaction mixture comprises a set of at least four different dNTPs one of which is a preselected detectable nucleotide, such that said elongated oligonucleotide comprises said detectable nucleotide.

7. A method according to claim 1 wherein said target molecule is a circular probe, said enzyme is a polymerase and said reaction mixture comprises a set of at least four different dNTPs, resulting in a rolling circle concatamer, wherein said reaction mixture further comprises at least one label probe that comprises said preselected detectable nucleotide.

8. A method according to claim 1 wherein said enzyme is a polymerase, said reaction mixture comprises a set of at least four different dNTPs that produces said elongation oligonucleotide, said reaction mixture further comprises at least one label probe that will bind to an elongated portion of said elongated oligonucleotide, and said method further comprises removing said target such that said label probe hybridizes to said elongation oligonucleotide.

9. A method according to claim 1, wherein said target sequence comprises a detection position, and said primer oligonucleotide comprises an interrogation base at the non-immobilized terminus, and said elongation only occurs if said interrogation base is complementary to said detection base in said assay complex.

10. A method according to claim 5, wherein said target sequence comprises a detection base, wherein either said primer oligonucleotide or said ligation probe comprises an interrogation base at the ligation site, wherein ligation only occurs if said interrogation base is complementary to said detection base in said assay complex.

11. The method of claim 1, wherein said detectable nucleotide is selected from the group consisting of 8-oxo-guanine and 5-aminouridine.

12. The method of claim 1, wherein said transition metal complex is osmium²⁺ (2,2′-bipyridine)₃.

13. The method of claim 1 wherein said electrode further comprises a self-assembled monolayer (SAM).

14. The method of claim 1 wherein said SAM comprises insulators comprising alkyl chains.

15. A method according to claim 1, wherein said target nucleic acid comprises DNA.

16. A method of detecting the presence of a target sequence in a sample comprising:
(a) providing a solid support comprising at least a first electrode having a first primer oligonucleotide immobilized thereon;
(b) hybridizing said sample with said primer oligonucleotide to form a hybridized nucleic acid;
(c) elongating said primer oligonucleotide using an enzyme to form an elongated oligonucleotide;
(d) contacting a solution comprising (i) a cationic electron donor of the formula comprising a transition metal ion and (ii) an anionic metal complex, to said elongated oligonucleotide under conditions in which said electron donor binds to said elongated oligonucleotide, transfers electrons to said electrode, and accepts electrons from said anionic metal complex,
(e) detecting said presence of said target nucleic acid from said detected electron transfer.

17. A method according to claim 16 further comprising:
(a) removing said target sequence after elongation; and
(c) adding at least one label probe that will bind to an elongated portion of said elongated oligonucleotide prior to said contacting step.

18. A method according to claim 16, wherein said cationic metal complex has the formula M(NH₃)₆³⁺, where M is selected from the group consisting of Ru and Co.

19. The method of claim 16, wherein M is Ru.

20. The method of claim 16, wherein said anionic metal complex comprises Fe(CN)₆³⁻.

21. The method of claim 16, wherein said anionic metal complex comprises a bipyridyl sulfonate metal complex.
